(19) United States
(12) Patent Application Publication
(10) Pub. No.: US 2026/0219545 A1
(43) Pub. Date: Jul. 30, 2026

(54) COLOR-TUNABLE UPCONVERSION-EMISSION SWITCH BASED ON COCRYSTAL-TO-COCRYSTAL TRANSFORMATION

(71) Applicant: Northwestern University, Evanston, IL (US)

(72) Inventors: Yu Wang, Evanston, IL (US); Huang Wu, Evanston, IL (US); James Fraser Stoddart, Evanston, IL (US)

(21) Appl. No.: 19/145,599

(22) PCT Filed: Jan. 4, 2024
(86) PCT No.: PCT/US2024/010243

§ 371 Date: Jul. 3, 2025

Related U.S. Application Data
(60) Provisional application No. 63/478,500, filed on Jan. 4, 2023.

(51) Int. Cl. G02F 1/355; C07C 25/22

(52) U.S. Cl. CPC G02F 1/3551; C07C 25/22; C07C 2602/02; C07C 2603/24

(57) ABSTRACT

Disclosed is a charge transfer cocrystal comprising an electron acceptor macrocycle and an electron donor and methods of making and using the same.

### CROSS-REFERENCE TO RELATED APPLICATIONS

The present application claims priority to U.S. Provisional Patent Application No. 63/478,500 that was filed Jan. 4, 2023. The entire contents of which are hereby incorporated by reference.

### BACKGROUND OF THE INVENTION

Cocrystal engineering, involving the assembly of two or more components into a highly ordered solid-state superstructure, has emerged as a strategy for tuning the photophysical properties of crystalline materials. The reversible co-assembly and disassembly of multicomponent cocrystals, as well as their reciprocal transformation in the solid state, remain challenging objectives.

### BRIEF SUMMARY OF THE INVENTION

In one aspect, the present disclosure provides a charge transfer cocrystal comprising an electron acceptor macrocycle and an electron donor. In some embodiments, the charge transfer cocrystal upconverts two or more absorbed photons. The charge transfer cocrystal may be color-tunable by substituting the electron donor for a second, different electron donor. The macrocycle can be a triangular macrocycle. For example, the macrocycle can be a naphthalenediimide-based triangular macrocycle (NDI-Δ) or a derivative thereof. The electron donor can be a substituted anthracene or a substituted naphthalene, including but not limited to 9, 10-dichloroanthracene (DCA) or 1-chloronaphthalene (CN). In particular embodiments, the cocrystal is DCA·NDI-Δ or CN·NDI-Δ.

In another aspect, the present disclosure provides a method for upconverting photons. The method can comprise irradiating a charge transfer cocrystal with photons at a first energy state, the charge transfer cocrystal comprising an electron acceptor macrocycle and an electron donor, whereby photons at a second energy state are emitted, the second energy state being higher than the first energy state.

In another aspect, the present disclosure provides a method for tuning color of light emission. The method can comprise providing a first charge transfer cocrystal comprising an electron acceptor macrocycle and a first electron donor. The method can further comprise at least partially substituting the first electron donor of the first charge transfer cocrystal with a second, different electron donor to produce a second charge transfer cocrystal. In the present method, the first charge transfer cocrystal when irradiated emits at a first wavelength and the second charge transfer cocrystal when irradiated emits a second, different wavelength. The method can further comprise substituting the second electron donor of the second charge transfer cocrystal with the first electron donor, thereby restoring the first charge transfer cocrystal.

In yet another aspect, the present disclosure provides a crystalline luminescent material, which comprises the charge transfer cocrystal as described herein. In some embodiments, the crystalline luminescent material is a wavelength tunable upconversion-emission material.

In yet another aspect, the present disclosure provides an apparatus comprising the charge transfer cocrystal or the crystalline luminescent material as described herein. The apparatus can be color-tunable. As non-limiting examples, the apparatus can be a color-tunable upconversion laser, a high-resolution display, or a multicolor imaging system. In a particular embodiment, the apparatus comprises a dual-color switch formed by two different charge transfer cocrystals as described herein. The two cocrystals can exhibit two-photon excited fluorescence.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1. Cocrystal-to-cocrystal transformation and structural formulas of NDI-Δ, DCA, and CN. Two cocrystals DCA·NDI-Δ and CN·NDI-Δ undergo reversible transformation based on the exchange of electron donors.

FIG. 2. Optical and fluorescence microscopic images of (a) the DCA·NDI-Δ cocrystal and (b) the CN·NDI-Δ cocrystal. (c) Solid-state UV-Vis absorption spectra of NDI-Δ, DCA, CN·NDI-Δ and DCA·NDI-Δ. (d) Solid-state fluorescence spectra of NDI-Δ, DCA, CN·NDI-Δ and DCA·NDI-Δ.

FIG. 3. Solid-state superstructure of the DCA·NDI-Δ cocrystal. (a) Plan view of a capped-sticks representation, showing the face-to-face packing between DCA and NDI-Δ. (b) Side-on view of a capped-sticks representation, demonstrating that two adjacent NDI-Δ macrocycles are stabilized by [C—H · · · O] hydrogen bonds. (c) Visualized intermolecular binding iso-surface between DCA and NDI-Δ. (d) Visualized intermolecular binding iso-surface between two adjacent NDI-Δ macrocycles. (e) Solid-state superstructure of the DCA·NDI-Δ cocrystal, illustrating that DCA molecules and NDI-Δ macrocycles stack to form a hexagonal superstructure.

FIG. 4. Solid-state superstructure of the CN·NDI-Δ cocrystal. (a) Plan view of a capped-sticks representation, showing the face-to-face packing between CN and NDI-Δ. (b) Side-on view of a capped-sticks representation, demonstrating that two adjacent NDI-Δ macrocycles are held together by [C—H · · · O] hydrogen bonds. (c) Visualized intermolecular binding iso-surface between CN and NDI-Δ. (d) Visualized intermolecular binding iso-surface between two adjacent NDI-Δ macrocycles. (e) Solid-state superstructure of the CN·NDI-Δ cocrystal, illustrating that CN and NDI-Δ assemble to afford a vertex-to-edge tiling pattern.

FIG. 5. The highest occupied molecular orbitals (HOMOs) and lowest unoccupied molecular orbitals (LUMOs) of DCA, DCA·NDI-Δ, CN·NDI-Δ and CN.

FIG. 6. Reversible cocrystal-to-cocrystal transformation. (a) Schematic illustration of cocrystal transformation between DCA·NDI-Δ and CN·NDI-Δ. The (I) represents the introduction of CN molecules by drop casting. The (II) involves the removal of CN molecules by solvent-vapor annealing. Fluorescence microscopic images of the (b) initial DCA·NDI-Δ film, (c) transformed CN·NDI-Δ film and (d) recovered DCA·NDI-Δ film. Fluorescence spectrum of the (e) initial DCA·NDI-Δ film, (f) transformed CN·NDI-Δ film and (g) recovered DCA·NDI-Δ film. (h) Powder X-ray diffraction patterns of the CN·NDI-Δ cocrystal, initial DCA·NDI-Δ film, transformed CN·NDI-Δ film, and recovered DCA·NDI-Δ film, showing that cocrystal transformation is reversible. (i) Raman spectra of the CN·NDI-Δ cocrystal, initial DCA·NDI-Δ film, transformed CN·NDI-Δ film, and recovered DCA·NDI-Δ film.

FIG. 7. (a) Two-photon excited fluorescence spectrum of the DCA·NDI-Δ cocrystal upon excited at 1000 nm. (b) The linear dependence between the upconversion-emission intensity and the square of the excitation laser power for the DCA·NDI-Δ cocrystal upon excited at 1000 nm. (c) Two-photon absorption spectrum of the DCA·NDI-Δ cocrystal measured with a constant laser power of 5.3 mW. (d) Two-photon excited fluorescence spectrum of the CN·NDI-Δ cocrystal upon excited at 800 nm. (e) The linear dependence between the upconversion-emission intensity and the square of the excitation laser power for the CN·NDI-Δ cocrystal upon excited at 700 nm. (f) Two-photon absorption spectrum of the CN·NDI-Δ cocrystal measured with a constant laser power of 5.3 mW. The insets are the two-photon microscopic images of the measured cocrystals.

FIG. 8. Reversible upconversion-emission switch. Two-photon microscopic images of the (a) initial DCA·NDI-Δ film, (b) transformed CN·NDI-Δ film and (c) recovered DCA·NDI-Δ film. The (I) represents the introduction of CN molecules by drop casting. The (II) involves the removal of CN molecules by solvent-vapor annealing. Two-photon excited fluorescence spectra of the (d) initial DCA·NDI-Δ film, (e) transformed CN·NDI-Δ film and (f) recovered DCA·NDI-A film. The linear dependence between the upconversion-emission intensity and the square of the excitation laser power for (g) the initial DCA·NDI-Δ film upon excited at 1000 nm, (h) the transformed CN·NDI-Δ film upon excited at 700 nm, and (i) the recovered DCA·NDI-Δ film upon excited at 1000 nm.

FIG. 9. Solid-state superstructures of DCA·NDI-Δ cocrystal. (a) Plan view of a capped-sticks representation showing the [Cl · · · π] and [Cl · · · H—C] interactions between DCA and NDI-Δ. (b) Side-on view of a capped-sticks representation demonstrating the [C—H · · · O] and [π · · · O═C] interactions between DCA and NDI-Δ.

FIG. 10. Solid-state superstructures of CN·NDI-Δ cocrystal. (a) Capped-sticks representation showing that one CN molecule interacts with two NDI-Δ macrocycles through [π · · · π] and [π · · · H—C] interactions. (b) Capped-sticks representation demonstrating the [C—H · · · O] and [π · · · O═C] interactions between CN and NDI-Δ.

FIG. 11. (a) The 1D columnar superstructure composed of NDI-Δ macrocycles along the a axis in CN·NDI-Δ cocrystal. (b) The vertex-to-edge tiling pattern in the b-c plane showing that the adjacent NDI-Δ macrocycles are held together by six CN molecules.

FIG. 12. (a) Capped-sticks representation of DCA molecule with a width of 6.30 Å and a length of 7.30 Å. (b) Capped-sticks representation of CN molecule with a width of 4.48 Å and a length of 4.85 Å.

FIG. 13. (a) Plan and (b) side-on views of the hexagonal superstructure of DCA·NDI-Δ cocrystal showing the intermolecular binding iso-surfaces between DCA and NDI-Δ molecules. Δκ inter (ρ)=0.003 a.u. Iso-surfaces are colored according to a BGR scheme over the range −0.05<sign(λ₂)ρ<+0.05 a.u.

FIG. 14. (a) Plan and (b) side-on views of the capped-stick representations of CN·NDI-Δ cocrystal showing the intermolecular binding iso-surfaces between CN and NDI-Δ molecules. Δκ inter (ρ)=0.003 a.u. Iso-surfaces are colored according to a BGR scheme over the range −0.05<sign(λ₂)ρ<+0.05 a.u.

FIG. 15. (a) Plan and (b) side-on views of the capped-stick representations of CN·NDI-Δ cocrystal showing the intermolecular binding iso-surfaces between two adjacent NDI-Δ molecules. Δκ inter (ρ)=0.003 a u. Iso-surfaces are colored according to a BGR scheme over the range −0.05<sign(λ₂)ρ<+0.05 a.u.

FIG. 16. Powder X-ray diffraction patterns of NDI-Δ crystals, DCA crystals, CN·NDI-A cocrystals, and DCA·NDI-Δ cocrystals. The powder X-ray diffraction spectra of CN·NDI-Δ and DCA·NDI-Δ cocrystals display new sets of diffraction peaks, which are different from those for individual donor and acceptor crystals, demonstrating the formation of new crystal superstructures.

FIG. 17. Optical microscopy pictures and corresponding fluorescence microscopy pictures of (a, b) DCA·NDI-Δ and (c, d) CN·NDI-Δ cocrystals.

FIG. 18. (a, b) SEM images of DCA·NDI-Δ cocrystals. (c) SEM-EDS Maps of DCA·NDI-Δ cocrystal showing all the component elements (C, N, O, Cl) well distributed within the cocrystal.

FIG. 19. (a, b) SEM images of CN·NDI-Δ cocrystals. (c) SEM-EDS Maps of CN·NDI-Δ cocrystal showing all the component elements (C, N, O, Cl) well distributed within the cocrystal.

FIG. 20. Raman spectra of NDI-Δ crystals, DCA crystals, CN·NDI-Δ cocrystals, and DCA·NDI-Δ cocrystals collected under excitation at 785 nm. Both the spectra of CN·NDI-Δ and DCA·NDI-Δ cocrystals include the naphthalene C═O stretching (1715 cm⁻¹), aromatic C═C and C—C stretching (1418, 1603 cm¹) peaks of NDI-Δ.

FIG. 21. The fluorescence decay curves for the (a) DCA·NDI-Δ and (b) CN·NDI-Δ cocrystals.

FIG. 22. (a) The DFT calculated binding energy between NDI-Δ and DCA in DCA·NDI-Δ cocrystal. (b) The DFT calculated binding energy between NDI-Δ and CN in CN·NDI-Δ cocrystal.

FIG. 23. Calculated one-photon absorption spectra of (a) DCA·NDI-Δ and (b) CN·NDI-Δ. Calculated two-photon absorption spectra of (c) DCA·NDI-Δ and (d) CN·NDI-Δ.

### DETAILED DESCRIPTION OF TH E INVENTION

The present disclosure relates to charge transfer cocrystals and methods thereof. In various embodiments, the present disclosure provides a color-tunable upconversion-emission switch based on the interconversion between two cocrystals. Examples of emissive cocrystals having different colors, composed of a macrocycle and different electron donors, have been obtained. The cocrystals can undergo reversible transformations on exchanging the electron donors. Benefiting from intermolecular charge transfer interactions, the cocrystals display superior two-photon excited upconversion emission. Accompanying the interconversion of the two cocrystals, their luminescent color changes between red and yellow, forming a dual-color upconversion-emission switch. This technology can involve precise control of cocrystal-to-cocrystal transformation and afford a reference for fabricating color-tunable nonlinear optical materials in the solid state.

The disclosed technology provides reversible cocrystal-to-cocrystal transformation based on the exchange of electron donors and an advanced strategy for the precise control of cocrystal transformation by exchanging their electron donors, realizing the dynamic modulation of superstructures in the crystalline state. The disclosed technology also provides for wavelength-tunable upconversion-emission materials in the crystalline state that may be used for the preparation of dynamic nonlinear optical materials with tunable emissions. These materials may be used in color-tunable upconversion lasers, high-resolution smart displays, or multicolor imaging reagents.

Organic cocrystals,¹⁻³ on account of their ease of preparation, well-defined superstructures and diverse functions, have generated considerable attention in the fields of supramolecular chemistry⁴⁻⁷, biomedical engineering^{8,9} and materials science¹⁰⁻¹². Cocrystals can be prepared by vapor-phase,¹¹ liquid-phase,^{13, 14} and solid-phase methods,¹⁵ all of which are low cost, convenient and feasible, as well as avoiding tedious covalent synthesis. With their ordered superstructures, organic cocrystals serve¹⁶⁻¹⁸ as promising candidates for investigating superstructure-property relationships Generally, cocrystals exhibit¹⁹ multifunctional behavior since they integrate the properties of their individual components. On some occasions, they display^{20, 21} innovative properties that differ from their precursors, such as metallic electrical conductivity,²² ambipolar charge transport,²³ room-temperature ferroelectricity,²⁴ nonlinear optical response,²⁵ and room-temperature phosphorescence²⁶. Moreover, with dynamic and reversible intermolecular noncovalent bonding interactions,^{27, 28} some of cocrystals exhibit²⁹⁻³² stimuli responsiveness, which makes them promising candidates for the assembly of smart materials. Co-assembly behavior during the formation of cocrystals, however, remains uncontrolled and unpredictable, making it difficult to customize specific cocrystals, not to mention realizing the interconversion between multicomponent cocrystals.

Endowing organic cocrystals with productive optical properties is³³⁻³⁷ a worthwhile goal for materials scientists. Crystalline luminescent materials with tunable fluorescent colors have been widely investigated,³⁸⁻⁴¹ because of their promising applications in visual displays, multiplexed bioimaging, information encryption and data storage. Their emission colors can be tuned by controlling the stoichiometric ratio of different precursors,⁴² altering the molecular packing modes,⁴³ and doping with additional components.⁴⁴ Among various luminescent materials, these with upconversion emission, which enable conversion⁴⁵ of lower-energy photons into higher-energy photons, have gained⁴⁶⁻⁴⁸ widespread interest. These materials possess⁴⁹ unique advantages including large penetration depths, low optical scattering, and high spatial resolution. Constructing wavelength-tunable upconversion-emission materials in the crystalline state is an attractive research objective on account of their potential applications as color-tunable upconversion lasers, high-resolution smart displays, multicolor imaging reagents and more into the bargain.⁵⁰⁻⁵³

In one aspect, the present disclosure provides a charge transfer cocrystal comprising an electron acceptor macrocycle and an electron donor.

In some embodiments, the charge transfer cocrystal upconverts two or more absorbed photons. The term “upconvert,” “upconversion,” or “photon upconversion” as used herein refers to a process in which absorption of two or more photons at an excitation wavelength by the cocrystal leads to emission of light at shorter wavelength (higher energy) than the excitation wavelength. For example, an upcoversion of an infrared light can led to emission of a visible light, or an upcoversion of red light can led to emission of a yellow light. As nonlimiting examples, the present charge transfer cocrystal can include organic molecules capable of achieving photon upconversion through triplet-triplet annihilation, including but not limited to polycyclic aromatic hydrocarbons (PAHs)

As demonstrated herein, the upconversion of photons by the present charge transfer cocrystals can be controlled by the chemical pairing of the electron acceptor macrocycle and the electron donor. In this process, a cocrystal can be chemically transformed into a different cocrystal with different chemical composition (electron donor-acceptor pair), which leads to different upconversion effects, including emission of lights of different wavelengths, as a result. Therefore, the present disclosure provides a means to tune the color of light emission via adjusting the chemical composition of the charge transfer cocrystals. In some embodiments, the present charge transfer cocrystal is color-tunable by substituting the electron donor for a second, different electron donor In such embodiments, a color-tunable upconversion-emission switch based on cocrystal-to-cocrystal transformation can be achieved.

In some embodiments, the macrocycle is a triangular macrocycle. In some embodiments, the macrocycle is a naphthalenediimide-based triangular macrocycle (NDI-Δ) or a derivative thereof. Suitable electron acceptor macrocycles include those described in U.S. Pat. Nos. 9,546,169, 10,745,418, 10,903,496, 11,155,554, and 11,560,644, and U.S. Patent Application Publication No. US 2021/0122767, all of which are incorporated herein by reference in their entireties.

Supramolecular tessellation of rigid triangular macrocycles in a 2D-plane results in the emergence materials where conductivity or photoconductivity may be tailored to particular applications. 2D semi-conductive materials, such as graphene, boron nitride, and transition metal dichalcogenides, are based on covalently interconnected atoms to form honeycomb 2D network patterns and are widely utilized in energy harvesting technologies and flexible electronics. Supramolecular tessellation of rigid triangular macrocycles similarly allows for preparation of semi-conductive materials. In this context, there is a combination of four parameters to be considered, namely (i) the rigid structure and the triangular shape of which can adopt different 2D tessellation packing motifs following the AT tiling principles, (ii) the confinement of electron donors inside the cavities leading to the formation of host-guest complexes which can facilitate CT, (iii) the greater electron-deficiency of triangular macrocycle, compared to that of the aromatic units, enhances the macrocycles electron affinity, and (iv) the intramolecular electron sharing between the aromatic units which offer potentially additional degrees of freedom for charge transport in 2D or 3D in tessellated packing patterns.

The rigid triangular macrocycle allows for tessellation in a two-dimensional plane. Suitably, the rigid triangular macrocycle may be formed from a functional n-system such as 1,4,5,8-naphthalene tetracarboxylic diimide (NDI). The rigid triangular macrocycle may be NDI-Δ. NDI-Δ includes (−)NDI-Δ or (+)NDI-Δ.

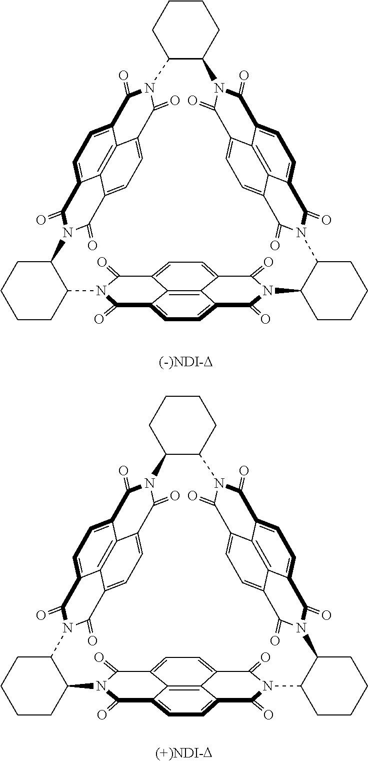

The structural and electronic properties of rigid shape-persistent NDI triangles display (i) electron sharing between three NDI units, leading to the observation of stepwise six-electron reductions, and (ii) anionic recognition properties. Incorporation into tessellated substructures allows for the development of compositions having the remarkable properties further described herein.

Rigid triangular macrocycles may be used to prepare charge transfer complexes with a n-electron or charge transfer donor The charge transfer donor may comprise a multi-ring aromatic structure, such as naphthalene, anthracene, or derivatives thereof. In some embodiments, the electron donor is a substituted anthracene or a substituted naphthalene. For example, the electron donor may be an anthracene substituted with one or more halogen or a naphthalene substituted with one or more halogen. The halogen can be, for example, chloro (Cl), bromo (Br), or Iodo (I). In some embodiments, the electron donor is an anthracene substituted with one or more Cl or a naphthalene substituted with one or more Cl. In other embodiments, the electron donor is 9, 10-dichloroanthracene (DCA) or 1-chloronaphthalene (CN).

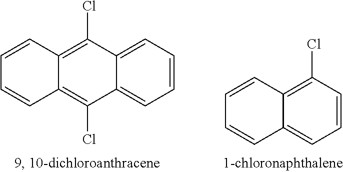 [SMILES: Clc1c2ccccc2c(Cl)c2ccccc12 ; Clc1cccc2ccccc12]

In some embodiments, the cocrystal is a cocrystal formed by DCA and NDI-Δ (herein referred to as DCA·NDI-Δ). In some embodiments, the cocrystal is a cocrystal formed by CN and NDI-Δ (herein referred to as CN·NDI-Δ).

In another aspect, the present disclosure provides a crystalline luminescent material, which comprises the charge transfer cocrystal as described herein. The luminescent material can include other one or more agents, such as colorants, preservatives, solvents, or stabilizers. The crystalline luminescent material can be color-tunable based on the chemical composition (electron donor-acceptor pair) of the charge transfer cocrystal and the corresponding upconversion effect (wavelength of the light emission). In some embodiments, they crystalline luminescent material is a wavelength tunable upconversion-emission material. As nonlimiting examples, the charge transfer cocrystal in the present crystalline luminescent material can be DCA·NDI-Δ, CN·NDI-Δ, or a combination thereof.

In another aspect, the present disclosure provides an apparatus, which comprises the charge transfer cocrystal or crystalline luminescent material described herein. The apparatus may be color-tunable, for example, due to chemical transformation of the electron donor-acceptor pairing of the charge transfer cocrystal and the corresponding color change of the light emission resulting from upconversion. The apparatus may further include other components, such as a housing, a power supply, irradiation source, and a control unit with programmable software. As nonlimiting examples, the present apparatus can be a color-tunable upconversion laser, a high-resolution display, or a multicolor imaging system. The multicolor imaging system can include other reagents.

In some embodiments, the present apparatus comprises a dual-color switch formed by two different charge transfer cocrystals described herein. As an example, the two cocrystals can exhibit two-photon excited fluorescence. As nonlimiting examples, the charge transfer cocrystal in the present apparatus can be DCA·NDI-Δ, CN·NDI-Δ, or a combination thereof.

In another aspect, the present disclosure provides a method for upconverting photons. The method comprises irradiating a charge transfer cocrystal with photons at a first energy state, the charge transfer cocrystal comprising an electron acceptor macrocycle and an electron donor, whereby photons at a second energy state are emitted, the second energy state being higher than the first energy state. In some embodiments, the charge transfer cocrystal as described herein may be used for the present method for upconverting photons.

The irradiation can have an excitation wavelength of about 700 nm to about 1000 nm. For example, the excitation wavelength can be about 700 nm, about 800 nm, about 900 nm, or about 1000 nm. The energy state of the emitted photons can be affected by the chemical composition of the charge transfer cocrystal, including the structures of the electron acceptor macrocycle and the electron donor. In some embodiments, the charge transfer cocrystal has an emission wavelength in the visible light range (e.g., 380-760 nm), such as red (620-750 nm), orange (590-625 nm), yellow (565-590 nm), green (500-565 nm), cyan (485-500 nm), blue (450-485 nm), and violet (380-450 nm).

As described herein, the emission wavelength (or color) as a result of the photon upconversion can be adjusted by changing the composition (electron donor-acceptor pairing) of the charge transfer cocrystal. In some embodiments, the macrocycle is a triangular macrocycle. For example, the macrocycle may be a naphthalenediimide-based triangular macrocycle (NDI-Δ) or a derivative thereof. The electron donor may be, for example, 9, 10-dichloroanthracene (DCA) or 1-chloronaphthalene (CN). As nonlimiting examples, the charge transfer cocrystal used in the present method for upconverting photons can be DCA·NDI-Δ, CN·NDI-Δ, or a combination thereof.

In another aspect, the present disclosure provides a method for tuning color of light emissions. The method comprises: providing a first charge transfer cocrystal comprising an electron acceptor macrocycle and a first electron donor, and at least partially substituting the first electron donor of the first charge transfer cocrystal with a second, different electron donor to produce a second charge transfer cocrystal, wherein the first charge transfer cocrystal when irradiated emits at a first wavelength, and wherein the second charge transfer cocrystal when irradiated emits a second, different wavelength.

In some embodiments, the charge transfer cocrystal as described herein may be used for the present method for tuning color of light emissions. As a result of electron donor substitution, the charge transfer cocrystal is transformed into a different cocrystal with a different charge transfer configuration, which can lead to different upconversion effect (e.g., emission wavelength) under irradiation. In some embodiments, the macrocycle for such cocrystal is a triangular macrocycle. For example, the macrocycle may be a naphthalenediimide-based triangular macrocycle (NDI-Δ) or a derivative thereof. Suitable electron donors include, but are not limited to, 9,10-dichloroanthracene (DCA) and 1-chloronaphthalene (CN). As nonlimiting examples, the charge transfer cocrystal used in the present method for tuning color of light emission can be DCA·NDI-Δ, CN·NDI-Δ, or a combination thereof.

The cocrystal-to-cocrystal transformation (e.g., from DCA·NDI-Δ to CN·NDI-Δ) can be carried out, for example, by drop casting or other suitable methods. For example, A CN solution (e.g., in methanol) can be dropped onto the surface of a DCA·NDI-Δ film. The droplet can then be evaporated, and the red DCA·NDI-Δ film transforms gradually into a yellow CN·NDI-Δ film. The transformed CN·NDI-Δ film can be restored to the DCA·NDI-Δ film, for example, using solvent-vapor annealing or other suitable methods. In some embodiments, the transformed CN·NDI-Δ film is exposed to the vapor of boiling CH₂Cl₂, the film can revert to the initial red DCA·NDI-Δ film.

In some embodiments, the first and second wavelengths are both in the visible light range. For example, one of cocrystals can emit yellow light and the other cocrystal can emit redlight when irradiated. In some embodiments, one of the first and second wavelengths is in the visible light range and the other is not.

In some embodiments, the method further includes substituting the second electron donor of the second charge transfer cocrystal with the first electron donor, thereby restoring the first charge transfer cocrystal. Thus, the cocrystal-to-cocrystal transformation can be controlled to switch between multiple emission wavelengths (e.g., multiple colors) as an output under irradiation. The transformation can be implemented in a programed manner to achieve a desired sequence of color output. Alternatively, the emission wavelength (e.g., color) can be tuned by transforming the charge transfer cocrystal (e.g., substituting one electron donor with another) according to a series of defined structures to achieve the desired emission wavelength or range of wavelengths. For example, the color tuning and programmed color output can be used in an optical instrument, an imaging system, a display, or a laser device.

In some embodiments, the method further comprises irradiating the second charge transfer cocrystal. In some embodiments, the method further comprises irradiating the first and the second charge transfer cocrystals. The light emission (e g., fluorescence) obtained from the irradiation can be use for color tuning or color output applications as described herein. The irradiation can have an excitation wavelength of about 700 nm to about 1000 nm. For example, the excitation wavelength can be about 700 nm, about 800 nm, about 900 nm, or about 1000 nm.

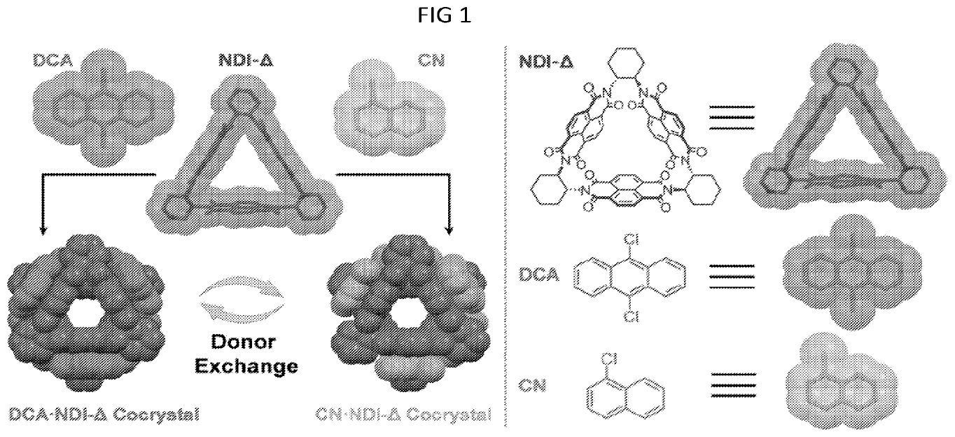

In particular embodiments, the present disclosure provides a reversible color-tunable upconversion-emission switch based on the interconversion of two charge transfer cocrystals (FIG. 1) With a naphthalenediimide-based triangular macrocycle (NDI-Δ) as the electron acceptor, and 9, 10-dichloroanthracene (DCA) or 1-chloronaphthalene (CN) as the electron donor, two cocrystals—namely, DCA·NDI-Δ and CN·NDI-Δ—have been obtained. These two cocrystals exhibit, respectively, quite different red and yellow fluorescence, indicating that even when using the same electron acceptor, a slight change in the molecular structure of the electron donor will cause a big difference in the properties of the resulting cocrystals. Single-crystal X-ray diffraction analysis reveal that the DCA·NDI-Δ cocrystal adopts a porous solid-state superstructure with a large guest-accessible void, while the CN·NDI-Δ cocrystal has a compact superstructure. The porous red-emissive DCA·NDI-Δ cocrystal can be transformed into the yellow-emissive CN·NDI-A cocrystal by introducing the CN component employing drop casting. This process can be reversed by the removal of the volatile CN molecules by solvent-vapor annealing. The reversible cocrystal-to-cocrystal transformation has been investigated by fluorescence microscopy, powder X-ray diffraction analysis, and Raman spectroscopy. Multiphoton microscopic experiments reveal that the two cocrystals exhibit strong two-photon excited fluorescence. Their red and yellow upconversion emissions can be switched accompanied by the reversible transformation between DCA·NDI-Δ and CN·NDI-Δ cocrystals, leading to the formation of a dual-color upconversion-emission switch. The present disclosure not only provides a rare, yet critical example of the cocrystal transformation based on the exchange of electron donor molecules, but it also affords a reference point for fabricating upconversion-emission switches and constructing dynamic nonlinear optical materials with tunable emissions in the solid state.

### ILLUSTRATIVE EMBODIMENTS

The following embodiments are illustrative and should not be interpreted to limit the scope of the claimed subject matter.

Embodiment 1. A charge transfer cocrystal comprising an electron acceptor macrocycle and an electron donor.

Embodiment 2. The charge transfer cocrystal of embodiment 1, wherein the charge transfer cocrystal upconverts two or more absorbed photons.

Embodiment 3. The charge transfer cocrystal of any one of the preceding embodiments, wherein the charge transfer cocrystal is color-tunable by substituting the electron donor for a second, different electron donor

Embodiment 4. The charge transfer cocrystal of any one of the preceding embodiments, wherein the macrocycle is a triangular macrocycle.

Embodiment 5. The charge transfer cocrystal of embodiment 4, wherein the macrocycle is a naphthalenediimide-based triangular macrocycle (NDI-Δ) or a derivative thereof.

Embodiment 6. The charge transfer cocrystal of any one of the preceding embodiments, wherein the electron donor is a substituted anthracene or a substituted naphthalene.

Embodiment 7. The charge transfer cocrystal of any one of the preceding embodiments, wherein the electron donor is an anthracene substituted with one or more halogen or a naphthalene substituted with one or more halogen.

Embodiment 8. The charge transfer cocrystal of any one of the preceding embodiments, wherein the electron donor is an anthracene substituted with one or more Cl or a naphthalene substituted with one or more Cl.

Embodiment 9. The charge transfer cocrystal of any one of the preceding embodiments, wherein the electron donor is 9, 10-dichloroanthracene (DCA) or 1-chloronaphthalene (CN).

Embodiment 10. The charge transfer cocrystal of any one of the preceding embodiments, wherein the cocrystal is DCA·NDI-Δ or CN·NDI-Δ.

Embodiment 11. A method for upconverting photons, the method comprising irradiating a charge transfer cocrystal with photons at a first energy state, the charge transfer cocrystal comprising an electron acceptor macrocycle and an electron donor, whereby photons at a second energy state are emitted, the second energy state being higher than the first energy state.

Embodiment 12. The method of embodiment 11, wherein the macrocycle is a triangular macrocycle.

Embodiment 13. The method of any one of embodiments 11-12, wherein the macrocycle is a naphthalenediimide-based triangular macrocycle (NDI-Δ) or a derivative thereof.

Embodiment 14. The method of any one of embodiments 11-13, wherein the electron donor is 9, 10-dichloroanthracene (DCA) or 1-chloronaphthalene (CN).

Embodiment 15. The method of any one of embodiments 11-14, wherein the cocrystal is DCA·NDI-Δ or CN·NDI-Δ.

Embodiment 16. A method for tuning color of light emission, the method comprising
providing a first charge transfer cocrystal comprising an electron acceptor macrocycle and a first electron donor; and
at least partially substituting the first electron donor of the first charge transfer cocrystal with a second, different electron donor to produce a second charge transfer cocrystal,
wherein the first charge transfer cocrystal when irradiated emits at a first wavelength; and wherein the second charge transfer cocrystal when irradiated emits a second, different wavelength.

Embodiment 17. The method of embodiment 16, further comprising substituting the second electron donor of the second charge transfer cocrystal with the first electron donor, thereby restoring the first charge transfer cocrystal.

Embodiment 18. The method of any one of embodiments 16-17, further comprising irradiating the second charge transfer cocrystal.

Embodiment 19. The method of any one of embodiments 16-18, further comprising irradiating the first and the second charge transfer cocrystals.

Embodiment 20. The method of any one of embodiments 16-19, wherein the macrocycle is a triangular macrocycle.

Embodiment 21. The method of any one of embodiments 16-20, wherein the macrocycle is a naphthalenediimide-based triangular macrocycle (NDI-Δ) or a derivative thereof.

Embodiment 22. The method of any one of embodiments 16-21, wherein one of the first and the second electron donor is 9, 10-dichloroanthracene (DCA) and the other is 1-chloronaphthalene (CN).

Embodiment 23. The method of any one of embodiments 16-22, wherein one of the first and the second cocrystal is DCA·NDI-Δ and the other is CN·NDI-Δ

Embodiment 24. A crystalline luminescent material comprising the charge transfer cocrystal of embodiment 1.

Embodiment 25. The crystalline luminescent material of embodiment 24, which is a wavelength tunable upconversion-emission material.

Embodiment 26. The crystalline luminescent material of any one of embodiments 24-25, wherein the cocrystal is DCA·NDI-Δ, CN·NDI-Δ, or a combination thereof.

Embodiment 27. An apparatus comprising the charge transfer cocrystal of embodiment 1 or the crystalline luminescent material of embodiment 24.

Embodiment 28. The apparatus of embodiment 27, which is color-tunable.

Embodiment 29. The apparatus of embodiment 27, which is a color-tunable upconversion laser, a high-resolution display, or a multicolor imaging system.

Embodiment 30. The apparatus of embodiment 27, comprising a dual-color switch formed by two different charge transfer cocrystals of embodiment 1.

Embodiment 31. The apparatus of embodiment 30, wherein the two cocrystals exhibit two-photon excited fluorescence.

Embodiment 32. The apparatus of embodiment 27, wherein the cocrystal is DCA·NDI-Δ, CN·NDI-Δ, or a combination thereof.

### EXAMPLES

The following Examples are illustrative and are not intended to limit the scope of the claimed subject matter.

### Growth of DCA·NDI-Δ and CN·NDI-Δ Cocrystals

The triangular macrocycle NDI-Δ,⁵⁴ containing three electron-deficient naphthalenediimide (NDI) units, serves⁵⁵ as a good electron acceptor. It enables binding of electron-rich moieties through outer-surface interactions,^{56, 57} forming charge transfer complexes. With a shape-persistent triangular geometry, NDI-Δ acts as a key building block for constructing⁵⁸ a variety of supramolecular architectures, e.g., single-handed helices, one-dimensional (1D) supramolecular nanotubes, and two-dimensional (2D) supramolecular tessellated patterns. Its diverse assembly behavior in the solid state indicates that it holds promise for constructing charge transfer cocrystals with exquisite superstructures. The π-conjugated NDI units^{42, 59, 60} also imbue NDI-Δ with a wealth of photophysical properties, making it a good candidate for building solid-state optical materials.

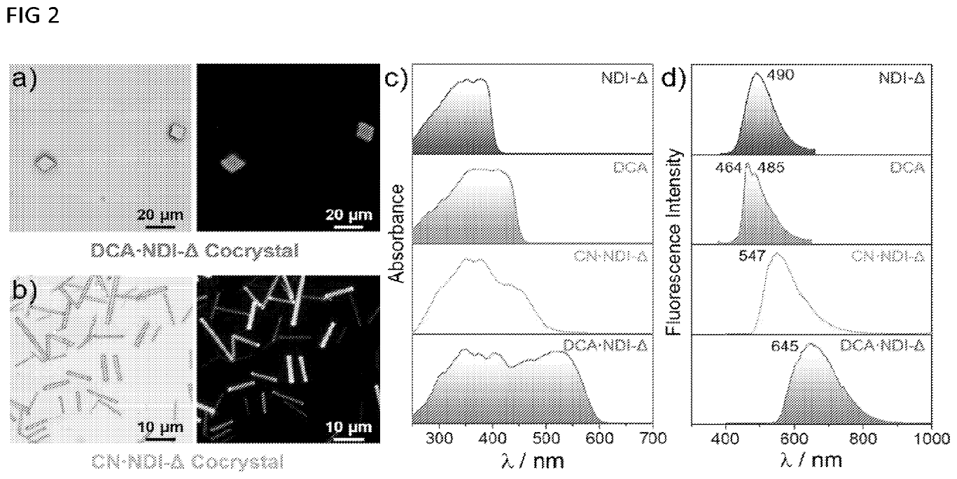

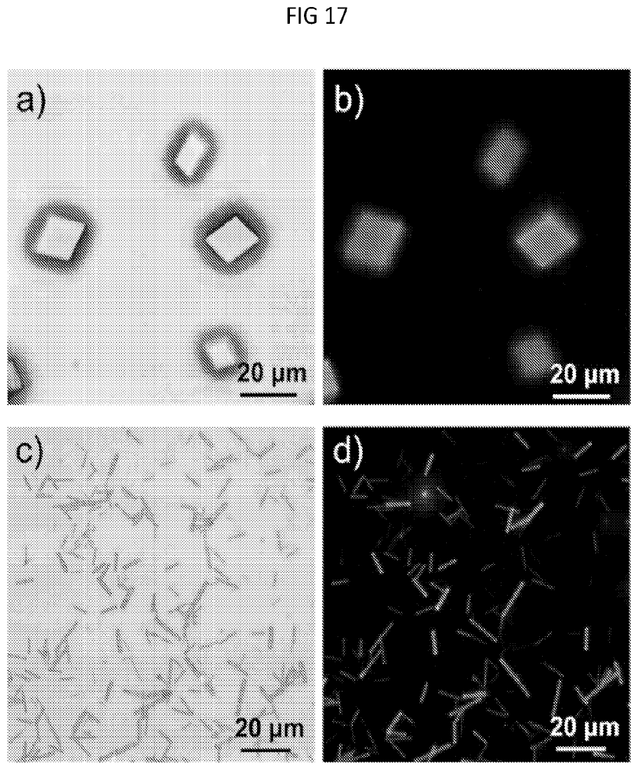

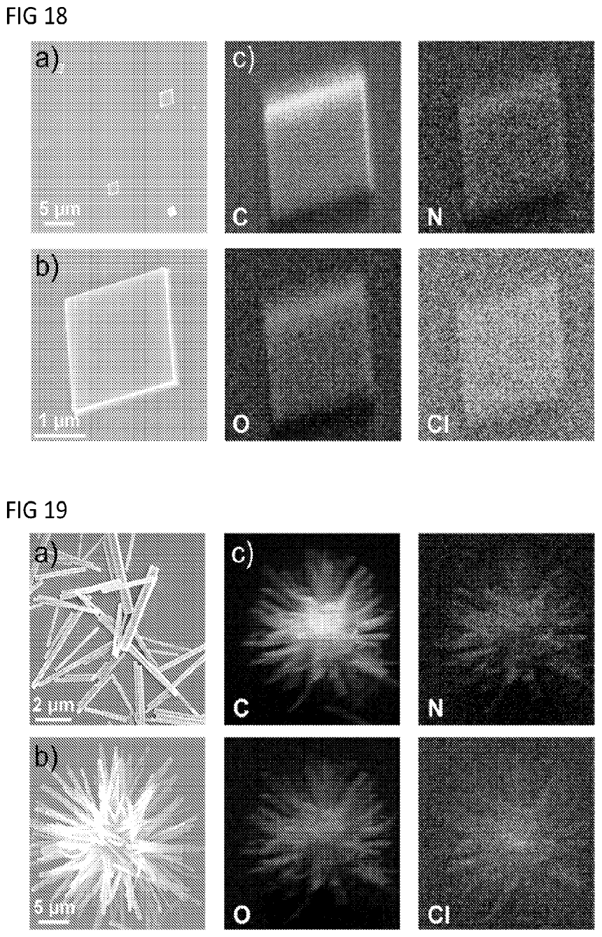

With NDI-Δ as the electron acceptor, two cocrystals with distinct emission colors based on different electron donors have been synthesized 9, 10-Dichloroanthracene (DCA), with good optical properties, ⁶¹ was selected as one of the electron donors. The DCA·NDI-Δ cocrystal was obtained by slow vapor diffusion of MeOH into a CH₂Cl₂ solution of NDI-Δ and DCA. The optical (FIGS. 2a and 17) and scanning electron microscopic (SEM, FIG. 18) images reveal that the DCA·NDI-Δ cocrystals have a 2D quadrangular shape with lengths ranging from a few to tens of micrometers. 1-Chloronaphthalene (CN) is⁶³ a colorless liquid at room temperature. Given its relatively smaller molecular size and volatility, it was employed as the other electron donor. The CN·NDI-Δ cocrystal was obtained by slow vapor diffusion of MeOH into a PhCl solution of NDI-Δ and CN. This cocrystal displays (FIGS. 2b and 19) ID rod-like morphology with lengths of up to a few tens of micrometers.

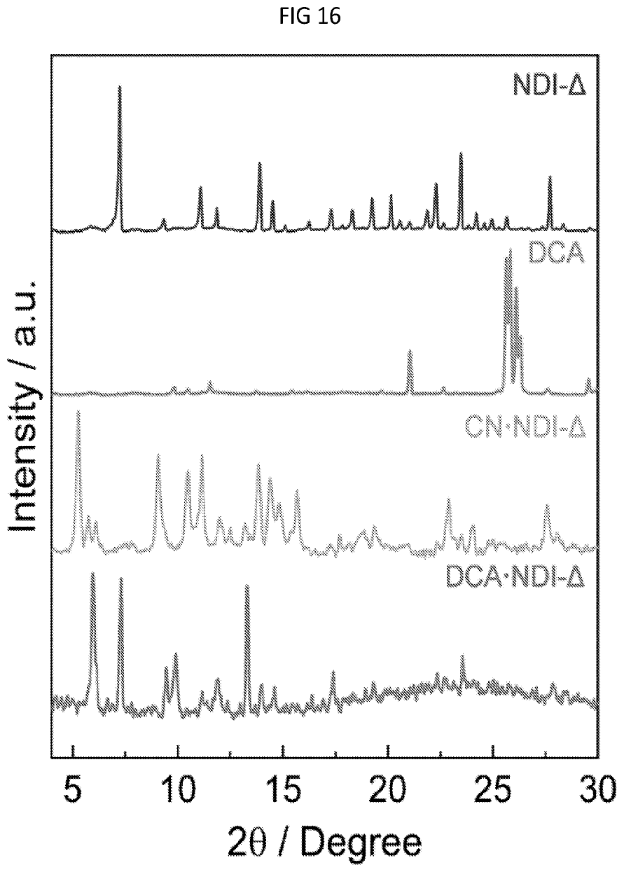

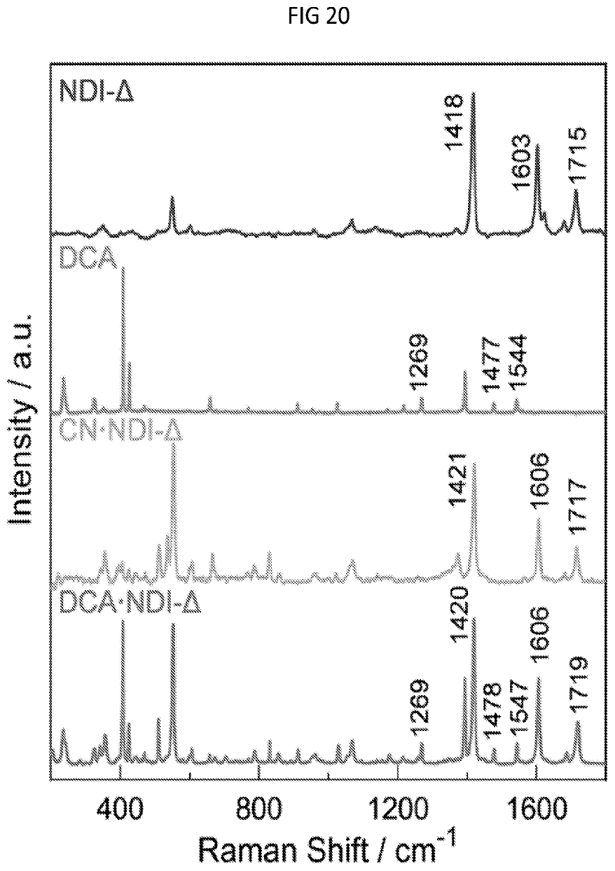

The formation of two cocrystals, DCA·NDI-Δ and CN·NDI-Δ, can be observed by the naked eye on account of the dramatic color change. NDI-Δ and DCA crystals are white and yellow, respectively, while the DCA·NDI-Δ cocrystal is red and the CN·NDI-Δ cocrystal is yellow. The formation of the DCA·NDI-Δ cocrystal has been confirmed by powder X-ray diffraction (PXRD) analysis, Raman spectroscopy, and SEM-equipped energy-dispersive X-ray spectroscopy (SEM-EDS). The PXRD pattern of the DCA·NDI-Δ shows (FIG. 16) a new set of diffraction peaks, which are different from those for the individual components, indicating the formation of a new solid-state superstructure. The Raman spectrum of DCA·NDI-Δ includes (FIG. 20) the characteristic peaks (1269, 1478, 1547 cm⁻¹) for DCA, and the vibrational peaks (1420, 1606, 1719 cm⁻¹) for NDI-Δ. The component elements of the DCA and NDI-Δ, including carbon, nitrogen, oxygen, and chlorine, are distributed (FIG. 18) homogeneously throughout the entire cocrystal according to the SEM-EDS analysis. All these results demonstrate the formation of a multicomponent DCA·NDI-Δ cocrystal based on charge transfer interactions between the electron-rich DCA and the electron-deficient NDI-Δ. The formation of the CN·NDI-Δ cocrystal has also been confirmed by PXRD patterns (FIG. 16), Raman spectroscopy (FIG. 20) and SEM-EDS analysis (FIG. 19). These results suggest that two macrocycle-based cocrystals have been obtained successfully by the co-assembly of NDI-Δ with two different electron donors.

### Photophysical Properties

With different electron-donating precursors, the DCA·NDI-Δ and CN·NDI-Δ cocrystals display distinctive optical properties. The fluorescence microscopic images indicate (FIGS. 2a and 1b) that the DCA·NDI-Δ and CN·NDI-Δ exhibit red and yellow luminescence, respectively. In order to elucidate their photophysical properties, solid-state UV-Vis absorption and fluorescence spectra were recorded. The UV-Vis absorption spectra reveal that the absorption bands of the NDI-Δ and DCA crystals are (FIG. 2c) in the region of 250-420 and 250-460 nm, respectively. The DCA·NDI-Δ cocrystal shows (FIG. 2c) a broad absorption band with a wavelength extended up to 593 nm, which is bathochromically shifted by over 130 nm in comparison with the absorption bands for NDI-Δ and DCA. This observation indicates that the cocrystal has a narrower bandgap than those of its processors. The solid-state absorption spectrum of the CN·NDI-Δcocrystal (FIG. 2c) reveals a wide absorption band ranging from 250 to 505 nm, which is −85 nm red-shifted compared to that of NDI-Δ. These red-shifted absorption bands of the DCA·NDI-Δ and CN·NDI-A cocrystals arise from intermolecular charge transfer interactions between electron donor and acceptor molecules. Optical bandgaps for the cocrystals can be calculated based on their absorption edges. The results disclose that the bandgap for DCA·NDI-Δ is 2.09 eV, which is narrower than that (2.46 eV) for CN·NDI-Δ The difference in bandgap may arise from the stronger electron-donating ability of DCA compared with that of CN.

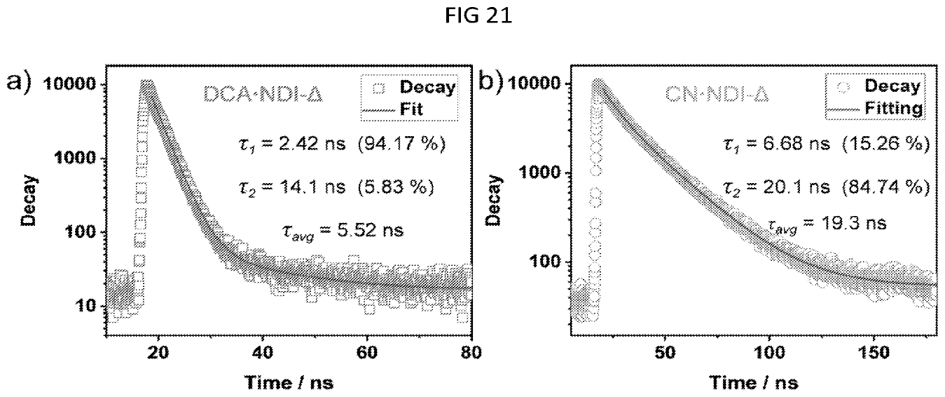

The DCA·NDI-Δ and CN·NDI-Δ cocrystals display red-shifted emission compared to their precursors, an observation which is in line with their absorption spectra. The emission band for the NDI-Δ crystal is (FIG. 2d) centered on 490 nm. Two emission peaks at 465 and 485 nm are observed (FIG. 2d) in the fluorescence spectrum of the DCA crystal. The DCA·NDI-Δ cocrystal exhibits (FIG. 2d) a deep-red emission at 645 nm, which is significantly red-shifted by 155 nm compared with that observed for the NDI-Δ crystal. This result indicates that the fluorescence of the cocrystal originates from the singlet excited state of a bimolecular species rather than from a monomeric one. The CN·NDI-Δ cocrystal shows (FIG. 2d) yellow fluorescence with an emission peak at 547 nm, demonstrating a red shift of 57 nm compared with that for the NDI-Δ crystal. The quantum yields (PLQYs, Φ_{F}) of the DCA·NDI-Δ and CN·NDI-Δ cocrystals are respectively 0.5 and 5.4%, values which are comparable with that (1.4%) for the electron acceptor NDI-Δ. Time-resolved fluorescence measurements demonstrate that DCA·NDI-Δ and CN·NDI-A display (FIG. 21) double-exponential fluorescence decay curves with average lifetimes of 5.52 and 19.3 ns, respectively. The DCA·NDI-Δ cocrystal displays more red-shifted absorption and emission in contrast with the CN·NDI-Δ cocrystal, indicating the stronger electronic communication between NDI-Δ and DCA. The difference in fluorescence of the two cocrystals suggests that cocrystallization serves as an effective tool to tune the optical properties of solid-state materials over a wide spectral range.

### Solid-State Superstructures

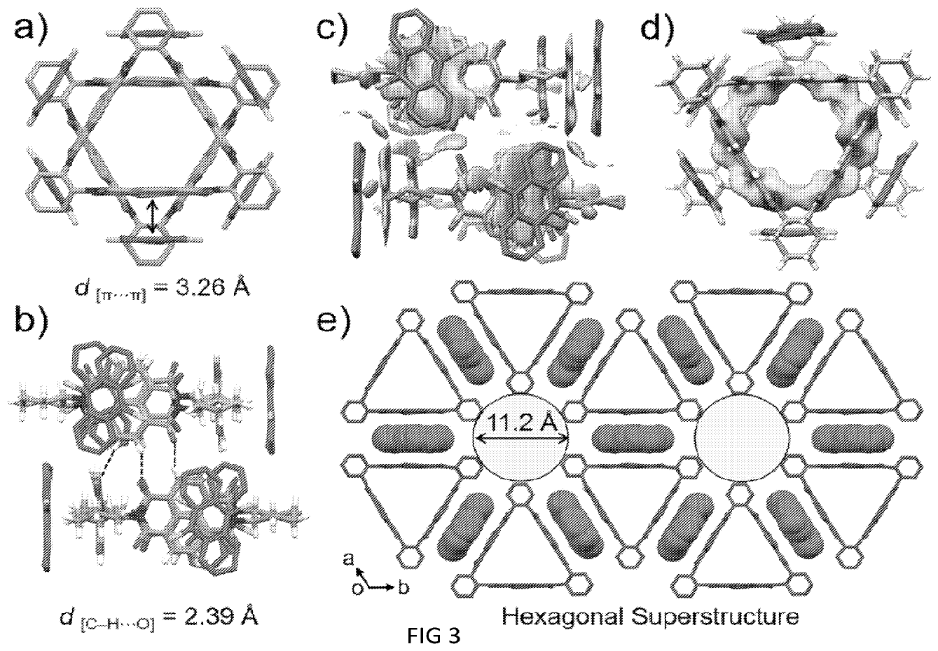

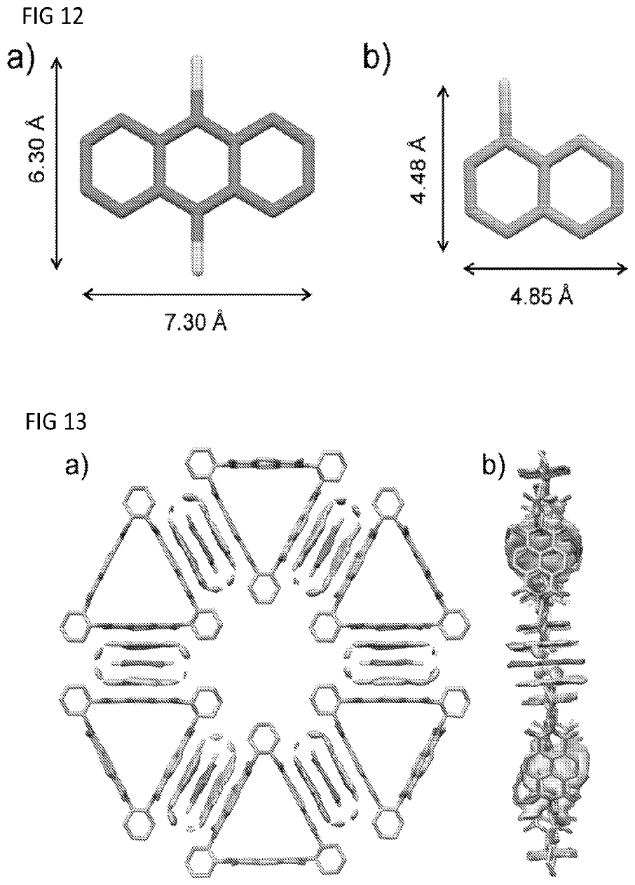

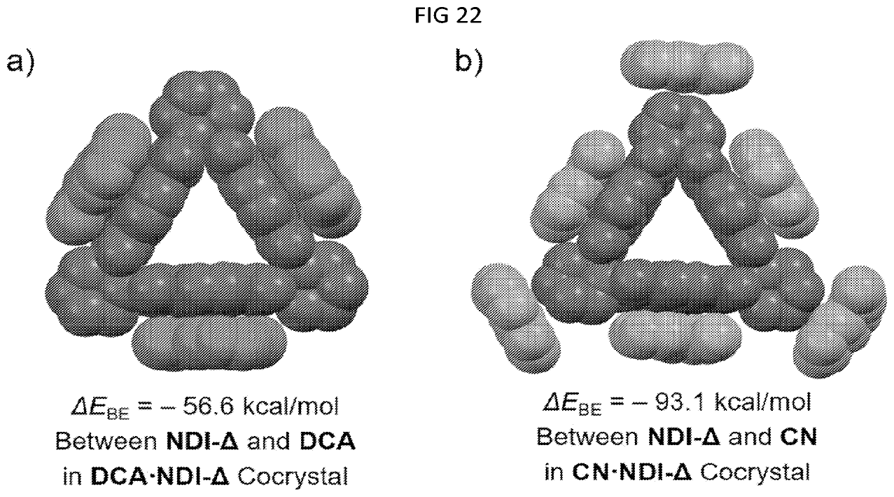

Single-crystal X-ray diffraction analyses were performed in order to investigate the intermolecular noncovalent interactions in the two donor-acceptor cocrystals and reveal their superstructure-photophysical property relationships. The DCA·NDI-Δ cocrystal adopts (Table 1) a trigonal R32 space group. Three DCA molecules stack (FIGS. 3a and 3c) face-to-face with three NDI units in NDI-Δ by means of [π · · · π] interactions with a distance of 3.26 Å. Additionally, the chlorine atoms in DCA have close contacts (FIG. 9) with the cyclohexano hydrogen atoms and the NDI unit in NDI-Δ with [Cl · · · H—C] and [Cl · · · π] distances of 2.79 and 3.30-3.36 Å, respectively. The calculated binding energy (|ΔE_{BE}|) between NDI-Δ and its surrounding three DCA molecules is (FIG. 22) 56.6 kcal/mol. Surrounded by DCA molecules, two adjacent NDI-A macrocycles stack (FIGS. 3b and 3d) in a coaxial manner with a rotation angle of 60°. The two NDI-Δ are stabilized by multiple [C—H · · · O] hydrogen bonds with a distance of 2.39 Å. Independent gradient model (IGM) analysis^{64, 65} provides a visual understanding of these noncovalent bonding interactions. Every DCA molecule interacts (FIGS. 3e and 13) with two NDI-Δ macrocycles sustained by multiple [π · · · π], [Cl · · · π] and [Cl · · · H—C] interactions. Consequently, six DCA molecules and six NDI-Δ macrocycles stack (FIGS. 3e and 13) around a vertex, forming a uniform hexagonal superstructure in the a-b plane. There is a pore in the center of the supramolecular hexagon with a diameter of 11.2 Å, leading to the formation of a porous superstructure with large guest-accessible voids of 36.3%.

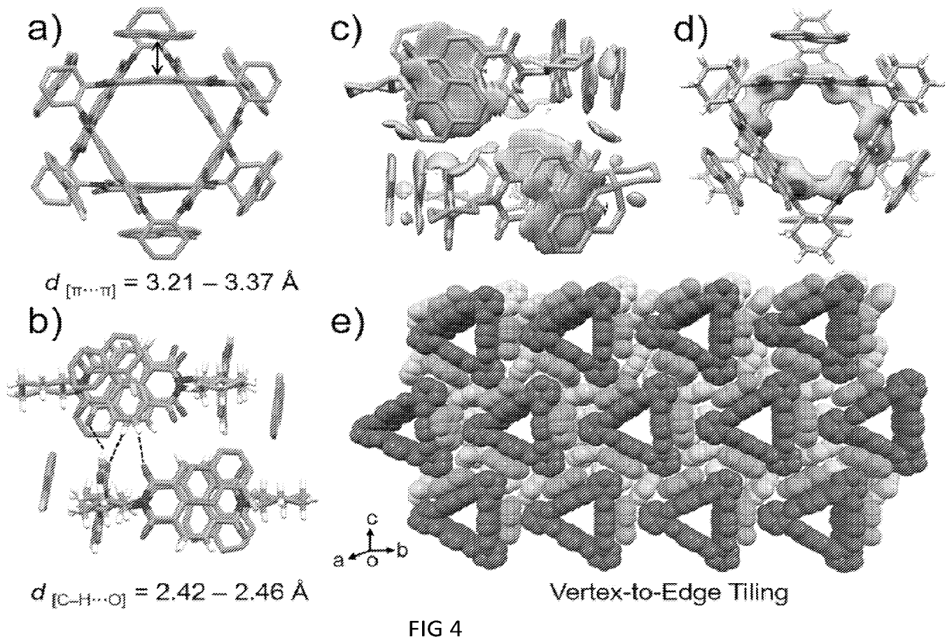

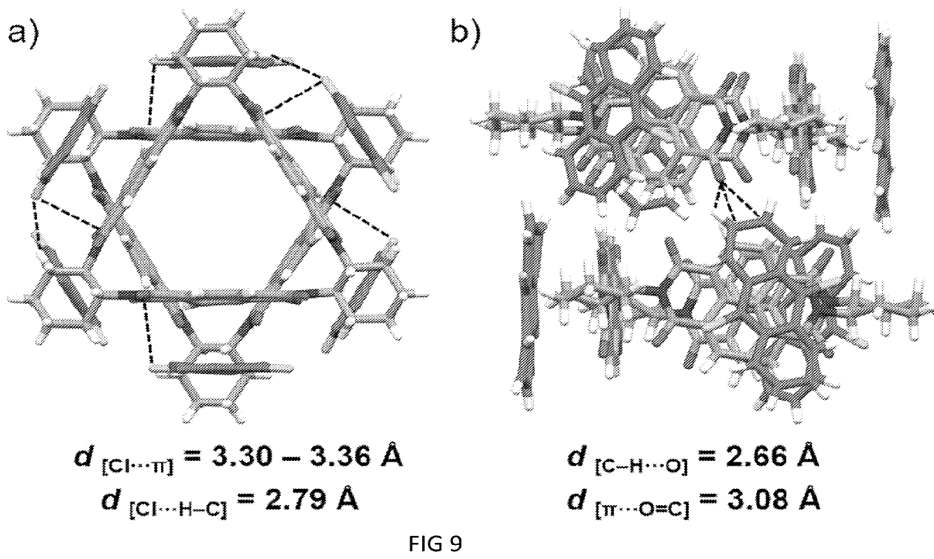

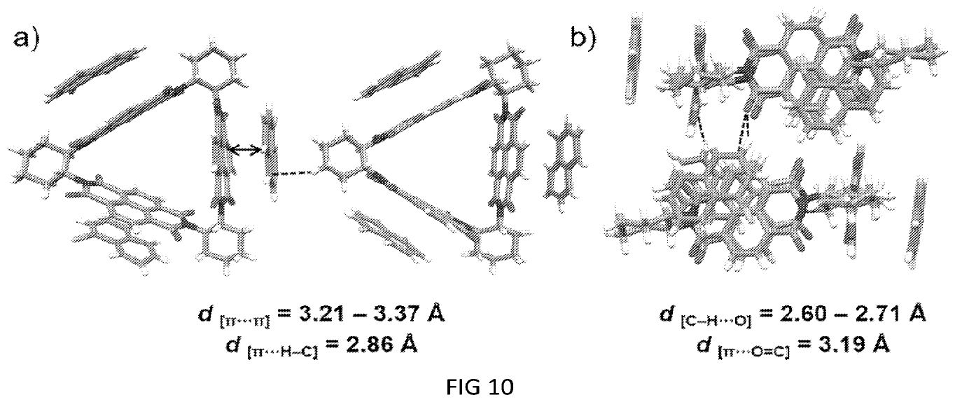

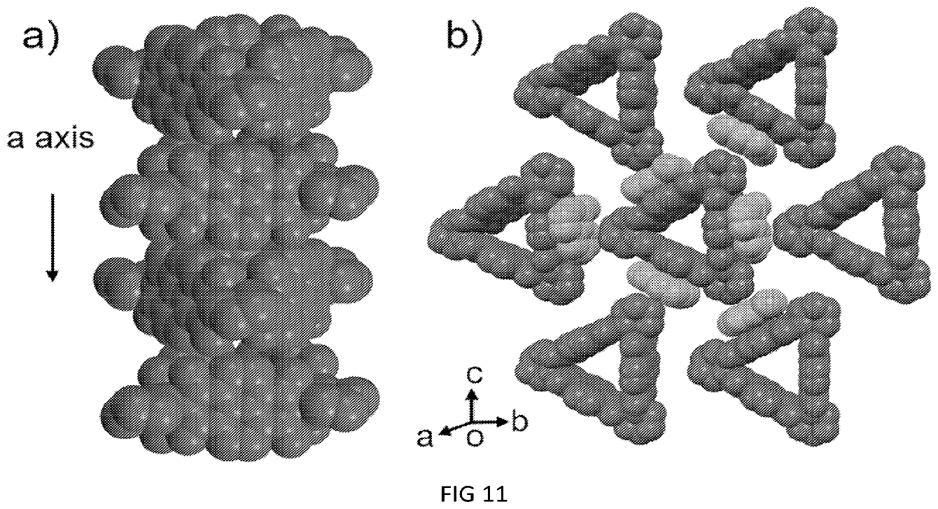

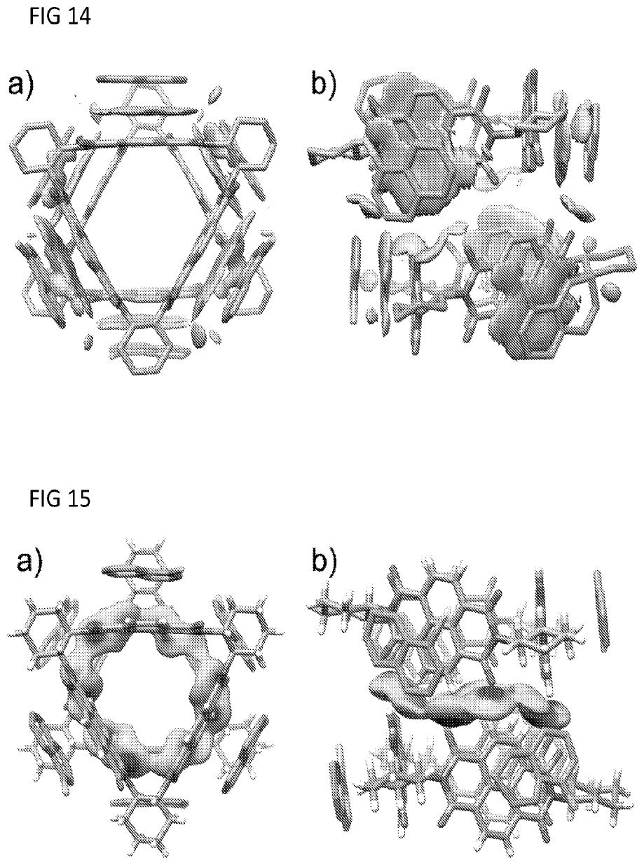

The solid-state superstructure of CN·NDI-Δ reveals that it crystallizes (Table 1) in a monoclinic P2₁ space group. The three NDI units in NDI-Δ interact (FIGS. 4a and 4c) with three CN molecules through face-to-face [π · · · π] interactions with distances ranging from 3.21 to 3.37 Å. Each NDI-Δ macrocycle is surrounded by another three CN molecules sustained by the [C—H · · · π] interactions (FIG. 10) between the cyclohexano hydrogen atoms in NDI-Δ and the π-plane of CN. The binding energy between NDI-Δ and its surrounding six CN molecules is calculated (FIG. 22) to be 93.1 kcal/mol. The NDI-Δ macrocycles stack into a ID column (FIG. 11) along the a axis, wherein adjacent NDI-Δ macrocycles connect (FIGS. 4b and 4d) with each other in a coaxial manner by means of multiple [C—H · · · O] hydrogen bonds with distances of 2.42-2.46 Å. These noncovalent interactions are visualized (FIGS. 14 and 15) by IGM analysis. NDI-Δ macrocycles in neighboring ID columns are held (FIG. 11) together by six CN molecules, forming a vertex-to-edge tiling pattern in the b-c plane. The lattice space between the vertex-to-edge tiling patterns is filled up (FIG. 4e) by several CN molecules, yielding a compact solid-state superstructure in the case of the CN·NDI-Δ cocrystal

### Frontier Molecular Orbitals Calculations

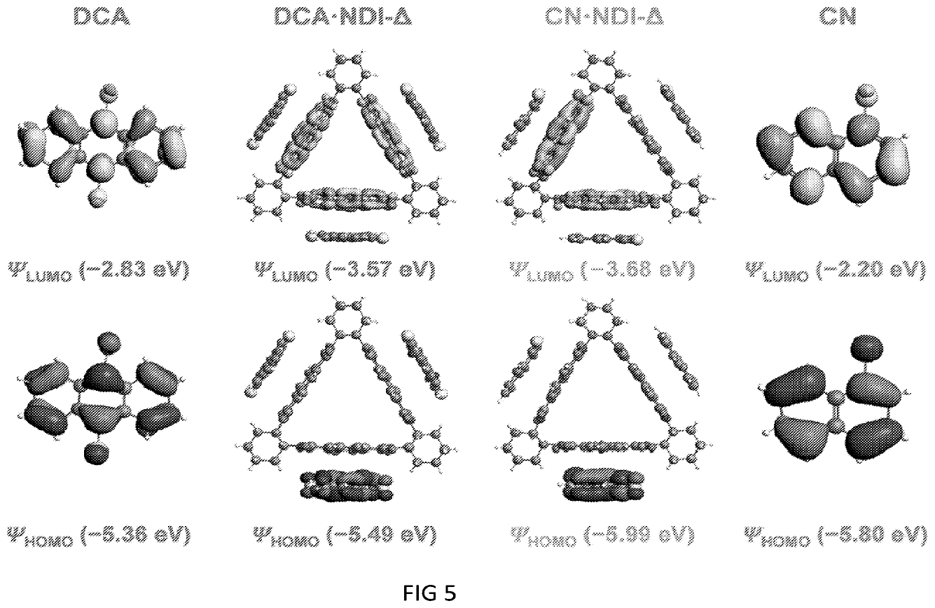

Density functional theory (DFT) calculations based on the solid-state superstructures of DCA·NDI-Δ and CN·NDI-Δ cocrystals were carried out to gain an in-depth understanding of the different photophysical properties of two cocrystals. The LUMOs of the DCA·NDI-Δ and CN·NDI-Δ cocrystals are (FIG. 5) concentrated on the electron-accepting NDI-Δ, and hence DCA·NDI-Δ and CN·NDI-Δ have similar LUMO energies of −3.57 and −3.68 eV, respectively. The HOMOs of the two cocrystals are localized on the electron-donating molecules. DCA possesses (FIG. 5) a higher HOMO energy of −5.36 eV compared to that (−5.80 eV) for CN, indicating that DCA shows stronger electron-donating ability. Accordingly, the DCA·NDI-Δ cocrystal has a higher HOMO energy of −5.49 eV compared to that (−5.99 eV) for CN·NDI-Δ. The HOMO-LUMO energy gaps (ΔE) of DCA·NDI-Δ and CN·NDI-Δ are (FIG. 5) calculated to be 1.92 and 2.31 eV, respectively, values which are in agreement with their optical bandgaps. The narrower energy gap of DCA·NDI-Δ, compared with that of CN·NDI-Δ, corresponds to the more red-shifted absorption and emission spectra of DCA·NDI-Δ. These results demonstrate that the bandgaps of the cocrystals are mainly determined by the HOMO of the electron donors and the LUMO of the electron acceptors. Their photophysical properties can be modulated effectively by tuning the energy levels of the donor and acceptor, which lays the foundation for constructing wavelength-tunable crystalline luminescent materials.

### Reversible Cocrystal-to-Cocrystal Transformation

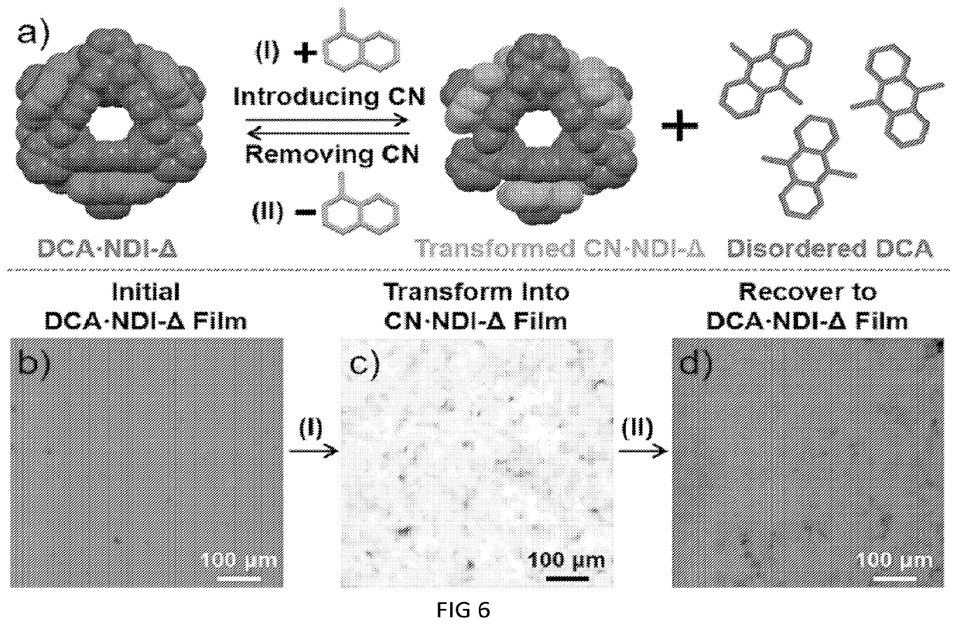

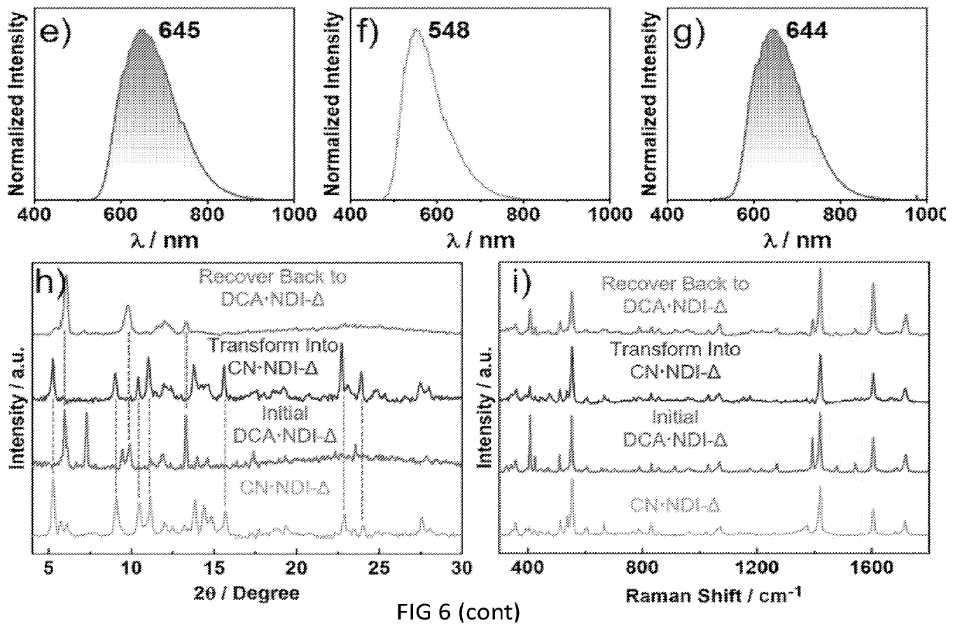

The porous solid-state superstructure of DCA·NDI-Δ provides a good platform to explore the dynamic cocrystal transformation (FIG. 6a). A DCA·NDI-Δ film was fabricated by dropping a suspension of the DCA·NDI-Δ cocrystal in MeOH onto a glass substrate, followed by air drying. When a CN solution in MeOH (CN/MeOH=1:9 v/v) was dropped onto the surface of the DCA·NDI-Δ film, the fluorescent color of the film underwent a significant change upon evaporation of the droplet. Its original red emission at 645 nm (FIGS. 6b and 6e) changed (FIGS. 6c and 6f) gradually to a yellow emission with a peak centered on 548 nm. The corresponding fluorescence spectrum is similar to that of the CN·NDI-Δ cocrystal, implying that the cocrystal converted from DCA·NDI-Δ to CN·NDI-Δ. This conversion was confirmed by PXRD analysis and Raman spectroscopy. After the DCA·NDI-Δ film transformed into the yellow-emissive one, the original PXRD peaks disappear (FIG. 6h), and a new set of diffraction peaks emerged. The resulting PXRD pattern is consistent (FIG. 6h) with that of the CN·NDI-Δ cocrystal, demonstrating the transformation from DCA·NDI-Δ to CN·NDI-Δ in the film. Raman spectroscopy reveals that the vibrational peaks for DCA·NDI-Δ film at 1269, 1478, 1547 cm-disappear (FIG. 6i) upon drop casting of the CN solution, and the resulting spectra correlate with that of the CN·NDI-Δ cocrystal. These observations demonstrate collectively that CN molecules are able to replace DCA molecules and cocrystalize with NDI-Δ in the solid state, leading to the conversion from the DCA·NDI-Δ to the CN·NDI-Δ film.

Remarkably, the transformed CN·NDI-Δ film can be reverted to the DCA·NDI-Δ film by solvent-vapor annealing⁶⁶. Upon exposure of the yellow-emissive transformed CN·NDI-Δ film to the vapor of boiling CH₂Cl₂, the fluorescence of the film recovers (FIGS. 6d and 6g) to the initial red emission with a peak at 644 nm, an observation which implies the restitution of the DCA·NDI-A film. In the PXRD pattern of the recovered red-emissive film, the characteristic PXRD peaks for the DCA·NDI-Δ cocrystal were observed (FIG. 6h), suggesting that the CN·NDI-Δ film changes back to the DCA·NDI-Δ film. When compared with the PXRD pattern of the initial DCA·NDI-Δ cocrystal, the PXRD peaks of the recovered DCA·NDI-Δ film are found to have broadened while several PXRD peaks have disappeared. This phenomenon may arise from the lower crystallinity of the recovered DCA·NDI-Δ obtained during the solid-state transformation, compared with that present in the initial DCA·NDI-Δ grown by solution crystallization. The Raman spectrum also confirmed the recovery of DCA·NDI-Δ film, wherein the vibrational peaks for the DCA·NDI-Δ cocrystal at 1269, 1478, 1547 cm⁻¹ reappear (FIG. 6i). These results indicate that these two cocrystals undergo reversible transformation upon introducing the CN component by drop casting and its removal by solvent-vapor annealing. Both the PXRD (FIG. 6h) and Raman spectroscopic (FIG. 6i) analyses reveal that only one kind of binary cocrystal—namely, DCA·NDI-Δ or CN·NDI-Δ—has been obtained after each transformation, indicating that the two binary cocrystals are more thermodynamically stable than the corresponding ternary products. This observation is the reason for the exchange of electron donors between DCA·NDI-Δ and CN·NDI-A cocrystals taking place after dropping the CN solution onto the surface of the DCA·NDI-Δ cocrystal, rather than the transformation into the ternary products. Accompanying the interconversion of the two cocrystals, the emission color of the film can undergo reversible changes between red and yellow, leading to the formation of a high-contrast dual-color fluorescent switch. This cocrystal interconversion can be repeated during several cycles. The fluorescence quantum yields of the recovered DCA·NDI-Δ and the transformed CN·NDI-Δ are 0.4 and 5.1%, respectively, after five cycles of cocrystal transformation. These values are comparable with the quantum yields of the initial DCA·NDI-Δ(0.5%) and CN·NDI-Δ (5.4%) cocrystals. The results indicate the good fatigue resistance for the interconversion between DCA·NDI-Δ and CN·NDI-Δ cocrystals. This cocrystal-to-cocrystal transformation based on the exchange of electron donors provides a practical strategy for the dynamic modulation of cocrystal superstructures and the fabrication of color-tunable crystalline luminescent materials.

Based on theoretical calculations and superstructures of the two cocrystals, we propose a possible process for the cocrystal transformation. DCA·NDI-Δ adopts a porous superstructure with a relatively high guest-accessible void fraction of 36.3%. When a CN solution in MeOH is dropped onto the surface of the DCA·NDI-Δ film, the small CN molecules (FIG. 12) diffuse into the voids in the DCA·NDI-Δ cocrystal by physical absorption. Since the total binding energy between NDI-Δ and CN is (FIG. 22) higher than that between NDI-Δ and DCA, CN replaces DCA gradually within the film. Consequently, the DCA·NDI-Δ film is transformed into the more stable CN·NDI-Δ film, leaving the DCA molecules distributed throughout the film in a disordered manner. CN is a colorless liquid at room temperature. Upon exposure of the transformed CN·NDI-A film to the vapor of boiling CH₂Cl₂, the volatile CN component is removed from the film by the rising CH₂Cl₂ vapor, on account of the fact that hot CH₂Cl₂ vapor accelerates the escape of CN molecules. The DCA and NDI-Δ molecules in the film rearrange under the CH₂Cl₂-vapor atmosphere, leading to the regeneration of the DCA·NDI-Δ film.

### Color-Tunable Upconversion Emission

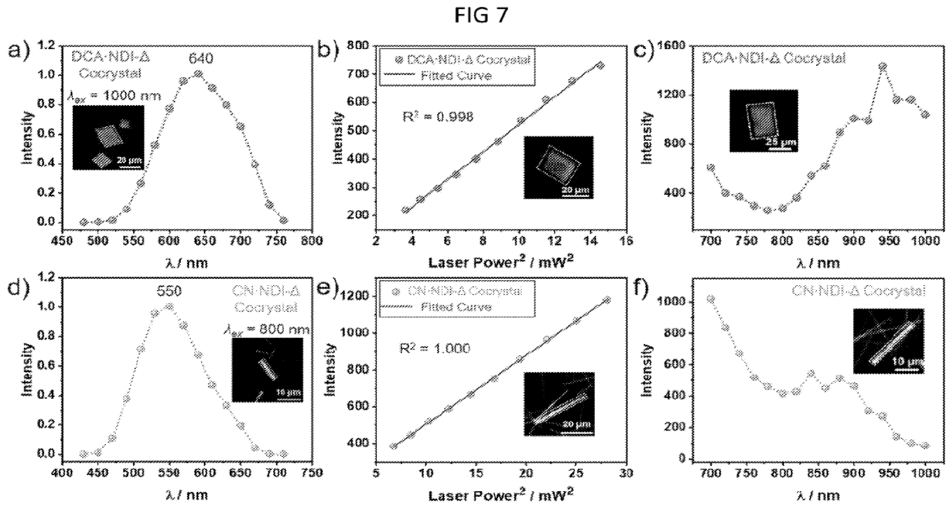

The DCA·NDI-Δ and CN·NDI-Δ cocrystals exhibit good nonlinear optical responses that arise from the intermolecular charge transfer interactions between electron donors and acceptors. The two-photon microscopic images reveal that the DCA·NDI-Δ cocrystal shows upconversion fluorescence upon excitation at 1000 nm, with an emission peak centered (FIG. 7a) on 640 nm, the value of which is analogous to its one-photon excited fluorescence at 645 nm. The emission intensity shows (FIG. 7b) a linear dependence on the square of incident laser power, suggesting that its upconversion emission originates from two-photon absorption.^{67, 68} The CN·NDI-Δ cocrystal also displays (FIG. 7e) a two-photon excited fluorescence with an emission band located at 550 nm (FIG. 7d), matching its one-photon excited fluorescence centered on 547 nm. The DCA·NDI-Δ and CN·NDI-Δ cocrystals exhibit red and yellow fluorescent colors upon two-photon excitation, respectively, observations that are essential for the construction of color-tunable upconversion-emission switches.

Considering the two-photon absorption is governed by different quantum-mechanical rules when compared with one-photon absorption, two-photon absorption spectra of two cocrystals were collected. In-situ two-photon imaging experiments have been performed, wherein the incident laser power was fixed at 5.3 mW, and the excitation wavelengths were changed from 700 to 1000 nm. The DCA·NDI-Δ cocrystal exhibits (FIG. 7c) a broad two-photon absorption spectrum with a maximum at 940 nm, whereas the strongest two-photon absorption of the CN·NDI-Δ cocrystal is observed (FIG. 7f) at 700 nm. These profiles demonstrate that the DCA·NDI-Δ cocrystal displays a more red-shifted two-photon absorption compared with that of the CN·NDI-Δ cocrystal, a phenomenon which is in agreement with that observed in their one-photon absorption spectra. Benefiting from the two-photon absorption properties of two cocrystals, their excitation wavelengths can be tuned from the UV-Vis into the NIR regions, making them promising NIR-absorbing materials.

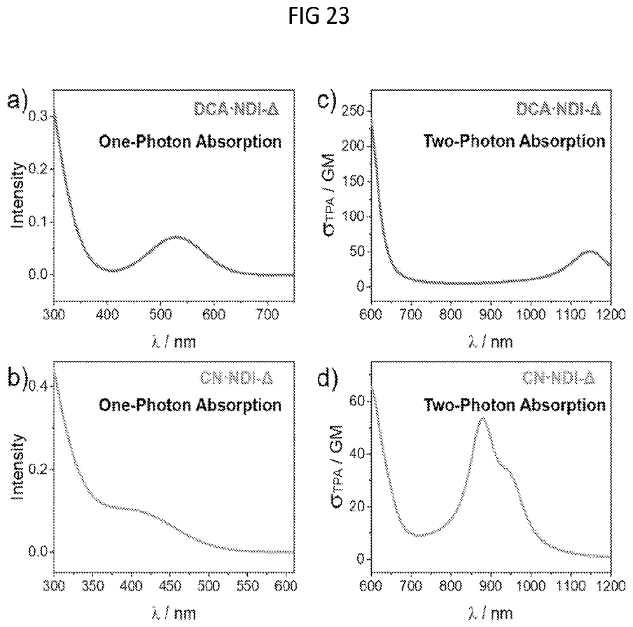

Time-dependent density functional theoretical (TDDFT) calculations were performed to investigate the two-photon absorption properties of the cocrystals in a wider wavelength range and gain in-depth understanding for their absorption cross-sections. The results reveal that DCA·NDI-Δ exhibits (FIG. 23) a maximum two-photon absorption cross-section (σ_{TPA}) of 239.2 GM at 600 nm, which is higher than that for CN·NDI-Δ (65.0 GM at 600 nm). The large two-photon absorption cross-sections of the two cocrystals may be attributed^{69, 70} to the efficient electron delocalization and the electron polarization induced by intermolecular charge transfer interactions between electron donors and acceptors. Notably, DCA·NDI-Δ displays (FIG. 23) a strong two-photon absorption band with a maximum G-RPA of 51.0 GM at 1148 nm in the NIR-II region, while CN·NDI-Δ shows a maximum σ_{TPA} of 53.5 GM at 880 nm in the NIR-I region. Compared to the CN·NDI-Δ cocrystal, DCA·NDI-Δ displays a more red-shifted two-photon absorption with bands reaching into the NIR-II region. These results indicate that the electron density of electron-donating molecules within the cocrystals plays a critical role in tuning the nonlinear optical properties of multicomponent crystals.

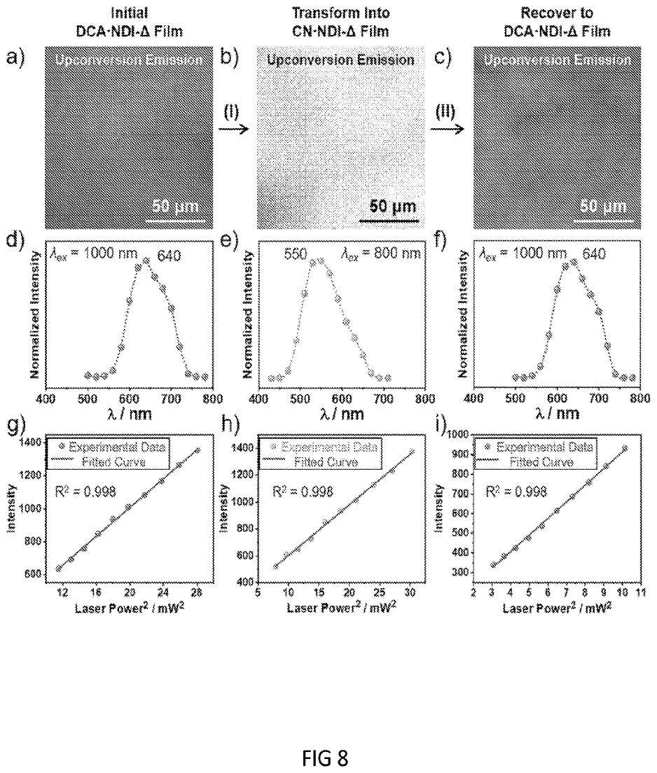

Taking advantage of the cocrystal-to-cocrystal transformation between DCA·NDI-Δ and CN·NDI-Δ, a dual-color upconversion-emission switch has been realized. Upon excitation at 1000 nm, the initial DCA·NDI-Δ film exhibits (FIGS. 8a, 8d, and 8g,) a red upconversion fluorescence with a peak centered on 640 nm. Upon drop casting of CN solution onto the surface of the DCA·NDI-Δ film, its upconversion-emission color changes from red to yellow. The corresponding upconversion-emission peak shifts (FIGS. 8b and 8e) from 640 to 550 nm, an observation which can be assigned to the transformation of DCA·NDI-Δ to CN·NDI-Δ in the film. The upconversion-emission intensity of the transformed CN·NDI-Δ film correlates (FIG. 8h) linearly with the square of the laser power, indicating that its two-photon absorption properties are retained. The yellow-emissive CN·NDI-Δfilm can be recovered to the initial red-emissive DCA·NDI-Δ film by solvent-vapor annealing. The maximum upconversion-emission wavelength of the film changes (FIGS. 8c and 8f) from 550 to 640 nm when the transformed CN·NDI-Δ film is subjected to the vapor of boiling CH₂Cl₂, suggesting the reconstruction of DCA·NDI-Δ. The upconversion-emission intensity of the recovered DCA·NDI-Δ film is proportional (FIG. 8i) to the square of the laser power. These results indicate that the color of two-photon excited fluorescence can be switched between red and yellow by the reversible transformation between DCA·NDI-Δ and CN·NDI-Δ cocrystals, leading to the formation of a dual-color upconversion-emission switch.

A color-tunable upconversion-emission switch based on cocrystal interconversion has been realized. With a naphthalenediimide-based molecular triangle NDI-Δ as the electron acceptor, two macrocycle-based cocrystals DCA·NDI-Δ and CN·NDI-Δ have been fabricated, which exhibit red and yellow fluorescence, respectively. The red-emissive DCA·NDI-Δ cocrystal can be transformed into the yellow-emissive CN·NDI-Δ cocrystal by drop casting of 1-chloronaphthalene, and can be recovered by the removal of volatile 1-chloronaphthalene through solvent-vapor annealing. Subsequently, a reversible cocrystal-to-cocrystal transformation based on the exchange of electron donors has been obtained. This work provides an advanced strategy for the precise control of cocrystal transformation by exchanging their electron donors, realizing the dynamic modulation of superstructures in the crystalline state. Notably, the two cocrystals exhibit good two-photon excited fluorescence, which can be attributed to donor-acceptor charge transfer interactions. By controlling the interconversion of two cocrystals, a high-contrast dual-color upconversion-emission switch has been fabricated, which paves the way for the construction of dynamic nonlinear optical materials with tunable emissions. These investigations afford an in-depth understanding of the controlled preparation and reciprocal transformation of multicomponent cocrystals, promoting the development of crystalline intelligent optical materials and their applications.

### EXPERIMENTS

### Materials, General Methods, and Instruments

All chemicals and solvents were purchased from commercial suppliers and used directly without further purification unless stated otherwise. (RRRRRR)-NDI-Δ—namely, NDI-Δ in this work—was synthesized and purified using methods⁷¹ reported previously in the literature. 9, 10-Dichloroanthracene (C₂₄H₁₂, 97%), defined as DCA, was purchased from Sigma-Aldrich. I-Chloronaphthalene (C₂₄H₁₂, 97%), defined as CN, was purchased from Sigma-Aldrich.

Optical and fluorescence microscopy images were captured with a Nikon LV150 microscope. Optical microscopy pictures were obtained in the bright field mode. Fluorescence microscopy images were obtained in the fluorescence mode. Scanning electron microscopy (SEM) images and energy-dispersive X-ray spectroscopy (EDS) elemental maps were captured on a SU8030 scanning electron microscope. Powder X-ray diffraction (PXRD) experiments were recorded on a STOE STADI MP powder diffractometer, which was equipped with an asymmetric curved Germanium monochromator (Cu K_{α1} radiation, λ=1.54056 Å) and a one-dimension silicon strip detector (MYTHEN2 1K from DECTRIS). The line-focused Cu X-ray tube was operated at 40 kV and 40 mA. The PXRD patterns were collected in reflection geometry mode at room temperature. UV-Vis absorption spectra were collected on a UV-3600 Shimadzu spectrophotometer equipped with an integrating sphere at room temperature. Fluorescence spectra and solid-state absolute photoluminescence quantum yields were carried out on a HORIBA FluoroMax-4 spectrometer equipped with an integrating sphere (HORIBA Quanta-<p). Raman spectra were recorded on a HORIBA LabRAM HR Evolution Confocal Raman instrument.

### Two-Photon Microscopy

The two-photon microscopic images were recorded on a Nikon AlR-MP⁺ Multiphoton Microscope equipped with a tunable Chameleon Vision titanium sapphire laser from 700 to 1000 nm. Two-photon absorption spectra were collected by in-situ two-photon imaging experiments wherein the images were recorded with the laser wavelengths ranging from 700 to 1000 nm and the laser power fixed at 5.3 mW. Two-photon excited fluorescence spectra were collected on a Leica DiveB SP8 Multiphoton Microscope equipped with a Spectra Physics InSight X3 laser. The corresponding two-photon microscopic images were recorded at a fixed laser wavelength and power, while the fluorescence detection wavelength was varied in the range of 380-780 nm.

### Density Functional Theory Calculation

The superstructures from the X-ray single crystals were used for the density functional theory⁷² (DFT) calculations in the Amsterdam Density Functional program⁷³ (ADF, version 2020.102). All-electron single point calculations were performed with the HSE06 functional, a triple zeta basis set⁷⁴ with a polarization function (TZP), and Grimme's third generation dispersion with Becke-Johnson damping. The molecular orbitals were visualized with ADFView.

We used second linear response time-dependent density functional theory (SLR-TDDFT) to compute the theoretical two-photon absorption spectra^{75, 76} and standard linear response TDDFT for the one-photon profiles. Although our calculations are “unrelaxed” for computational efficiency, they agree with relaxed calculations to a very good degree-relaxed simulations are quite computationally demanding for the dimers considered in this work. The PBE0 exchange-correlation functional⁷⁷ was employed in our TDDFT applications. The Tamm-Dancoff method and the basis set/pseudo-potential SBKJC were used as well. The Davidson threshold used here is 10⁻². The two-photon absorption spectra are computed through the well-known sum-over-states formula^{78, 79} with an intermediate state broadening factor of 0.1 eV and line-shape broadening of 0.2 eV. As a result of the very high number of unoccupied orbitals, for the DCA·NDI-Δ calculations, we ignored virtual orbitals with energies larger than 6.87 eV with respect to the highest-occupied orbital.

### Synthetic Protocols

### (1) Preparation of the DCA·NDI-Δ Cocrystals

The NDI-Δ (10.4 mg, 10 μmol) macrocycle and DCA (2.5 mg, 10 μmol) were dissolved (Scheme 1) in CH₂Cl₂ (10 mL). The solution was passed through with a 0.22-μm syringe filter to remove the insoluble substances. With slow vapor diffusion of MeOH into the filtrate, high-quality red quadrangle-shaped DCA·NDI-Δ cocrystals formed during the period of one week.

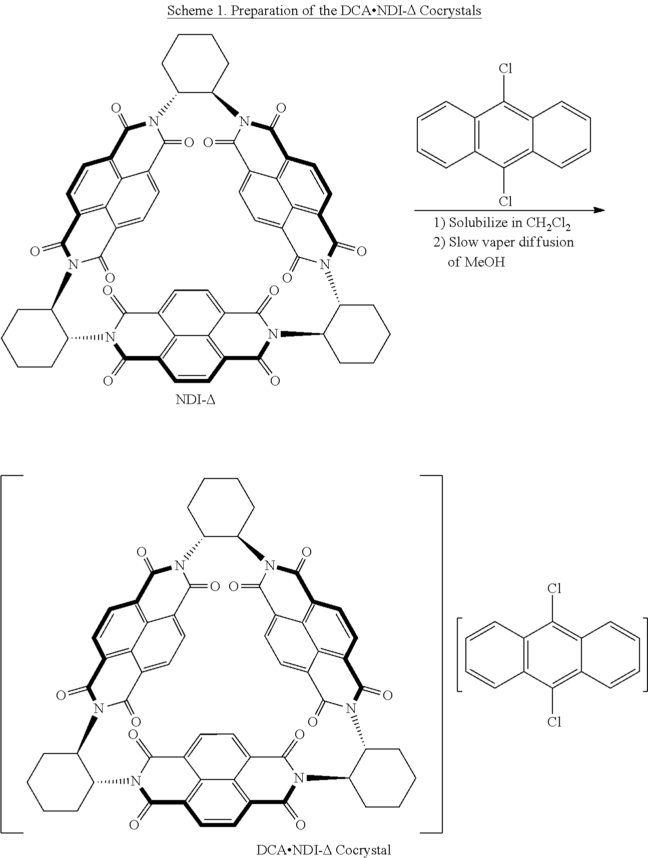

### (2) Preparation of the CN·NDI-Δ Cocrystals

The NDI-Δ (10.4 mg, 10 μmol) macrocycle was dissolved (Scheme 2) in PhCl (10 mL). Then the solution was passed through with a 0.22-μm syringe filter to remove insoluble substances. The CN (100 μl) liquid was added to the PhCl solution of NDI-Δ. With slow vapor diffusion of MeOH into the solution of NDI-Δ and CN, high-quality yellow needle-shaped CN·NDI-Δ cocrystals formed after one week

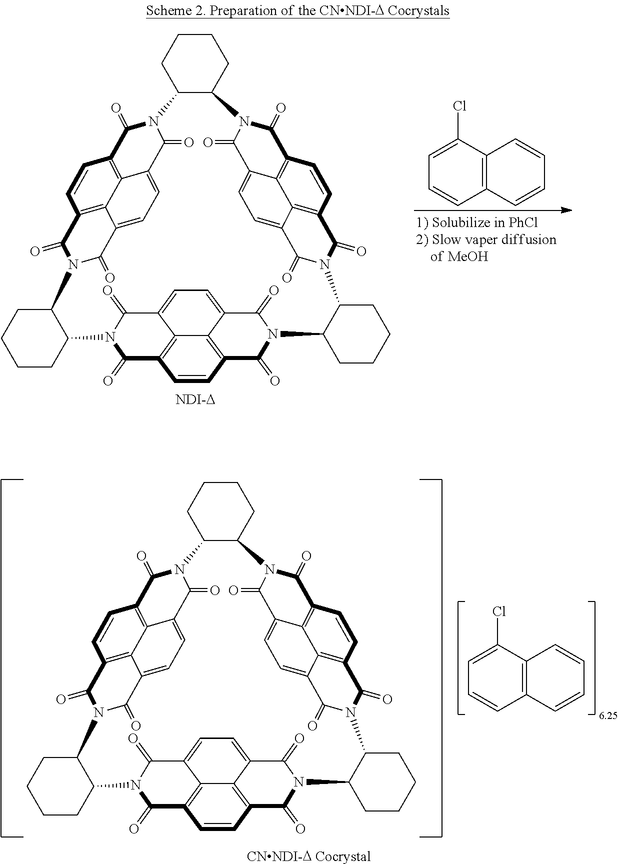

### Crystallographic Characterization

### (1) DCA·NDI-Δ Cocrystals

(a) Methods. DCA·NDI-Δ cocrystals suitable for single-crystal X-ray crystallography, were prepared by slow vapor diffusion of MeOH into a CH₂Cl₂ solution of DCA and NDI-Δ. A suitable crystal was selected and the crystal was mounted on a MITIGEN holder with Paratone oil on a XtaLAB Synergy R, DW system, HyPix diffractometer. The crystal was kept at 100.00(10) K during data collection. Using Olex2,⁸⁰ the superstructure was solved with the ShelXT⁸¹ structure solution program using Intrinsic Phasing and refined with the XL⁸² refinement package using Least Squares minimisation.

(b) Refinement details. The enhanced rigid-bond restraint (SHELX keyword RIGU) as well as restraints on similar amplitudes separated by less than 1.7 Å was applied for the disordered dichloroanthracene.⁸³ Distance restraints were also applied on the dichloroanthracene.

(c) Solvent treatment details. The solvent masking procedure as implemented in Olex2 was used to remove the electronic contribution of solvent molecules from the refinement. As the exact solvent content is not known, only the atoms used in the refinement model are reported in the formula here. Total solvent accessible volume/cell=4827.3 Å³ [36.3%] Total electron count/cell=822.2.

### (2) CN·NDI-Δ Cocrystals

(a) Methods. High quality CN·NDI-Δ cocrystals suitable for single-crystal X-ray crystallography, were prepared by slow vapor diffusion of MeOH into a PhCl solution of CN and NDI-Δ. A suitable crystal was selected and the crystal was mounted on a MITIGEN holder with Paratone oil on a XtaLAB Synergy R, DW system, HyPix diffractometer. The crystal was kept at 175.00(10) K during data collection. Using Olex2,⁸⁰ the superstructure was solved with the XT⁸¹ structure solution program using Intrinsic Phasing and refined with the XL⁸² refinement package using Least Squares minimisation.

(b) Refinement details. Distance restraints were imposed on the 1-chloronaphthalene and PhCl molecules. The enhanced rigid-bond restraint (SHELX keyword RIGU) was applied globally.⁸³ Restraints on similar amplitudes separated by less than 1.7 Å were also imposed on the chloronaphthalene.

(c) Solvent treatment details. N/A.

TABLE 1
Crystallographic Data for DCA•NDI-Δ and CN•NDI-Δ

| Complex | DCA•NDI-Δ | CN•NDI-Δ |
|---|---|---|
| Empirical formula | C₇₄H₅₀Cl₂N₆O₁₂ | C₁₃₀H₉₂Cl_{7.5}N₆O₁₂ |
| Formula weight | 1286.10 | 2195.97 |
| T/K | 100.01(10) | 175.00(10) |
| Crystal system | trigonal | monoclinic |
| Space group | R32 | P2₁ |
| a/Å | 24.6999(6) | 15.9639(4) |
| b/Å | 24.6999(6) | 19.5835(5) |
| c/Å | 25.1460(9) | 33.6729(8) |
| α/° | 90 | 90 |
| β/° | 90 | 95.758(2) |
| γ/° | 120 | 90 |
| V/Å³ | 13285.9(8) | 10474.0(5) |
| Z | 6 | 4 |
| ρ_{calcd}/g cm⁻³ | 0.964 | 1.393 |
| μ/mm⁻¹ | 1.077 | 2.415 |
| F (000) | 3996 | 4550 |
| goodness-of-fit on F² | 1.402 | 1.209 |
| R₁ [I > 2σ (I)] | 0.1289 | 0.1169 |
| wR₂ [all data] | 0.3274 | 0.3557 |
| CCDC No. | 2212309 | 2212308 |

### IGM Calculations

Independent gradient model (IGM) analysis⁸⁴ is an approach based on promolecular density (an electron density model prior to molecule formation) to identify and isolate intermolecular interactions. Strong polar attractions and van der Waals contacts are visualized as an iso-surface with blue and green colors, respectively. Single crystal superstructures were used as input files. The binding surface was calculated using the Multiwfn 3.8 program⁸⁵ through function 20 (visual study of weak interactions) and visualized by Chimera software.⁸⁶

The binding energies were computed as ΔE_{BE}=products−reactants, where the products are the single crystal systems and the reactants are the CN, DCA and NDI-Δmolecules taken from the single crystals. In the DCA·NDI-Δ system, the energy of one DCA was used as all three DCA surrounding each NDI-Δ are in the same environment. In the CN·NDI-Δ system, there are two CN environments, so one facial and one corner CN was computed. Note that the distinction between the two CN environments has little energetic difference in comparison with just one on the total binding energy, but was done for completeness.

### Cocrystal-to-Cocrystal Transformation

The DCA·NDI-Δ cocrystal was dispersed in a MeOH solution. Upon adding the suspension of DCA·NDI-Δ cocrystal onto a glass substrate, the DCA·NDI-Δ film was obtained after air drying. The transformation from DCA·NDI-Δ film to CN·NDI-Δ was realized by employing a drop casting method. A CN solution in MeOH (CN/MeOH=1:9 v/v) was dropped onto the surface of the DCA·NDI-Δ film. The droplet was evaporated after 0.5 h, and the red DCA·NDI-Δ film transforms gradually into the yellow CN·NDI-Δ film. The transformed CN·NDI-Δ film can be recovered to the DCA·NDI-Δ film using solvent-vapor annealing. Upon exposure of the transformed CN·NDI-Δ film to the vapor of boiling CH₂Cl₂, the film reverted to the initial red DCA·NDI-Δ film after 2 h.

### REFERENCES

1 Aitipamula, S.; Banerjee, R.; Bansal, A. K.; Biradha, K.; Cheney, M. L.; Choudhury, A. R.; Desiraju, G. R.; Dikundwar, A. G.; Dubey, R.; Duggirala, N.; et al. Polymorphs, Salts, and Cocrystals: What's in a Name?Cryst. Growth Des. 2012, 12, 2147-2152.
2 Goetz, K. P.; Vermeulen, D.; Payne, M. E.; Kloc, C.; McNeil, L. E; Jurchescu, O. D Charge-Transfer Complexes: New Perspectives on an Old Class of Compounds. J. Mater. Chem. C 2014, 2, 3065-3076.
3 Jiang, H.; Hu, P.; Ye, J., Zhang, K. K.; Long, Y.; Hu, W.; Kloc, C. Tuning of the Degree of Charge Transfer and the Electronic Properties in Organic Binary Compounds by Crystal Engineering. A Perspective. J. Mater. Chem. C 2018, 6, 1884-1902.
4 Little, M. A.; Briggs, M. E.; Jones, J. T. A.; Schmidtmann, M.; Hasell, T.; Chong, S. Y.; Jelfs, K. E., Chen, L., Cooper, A. 1. Trapping Virtual Pores by Crystal Retro-Engineering. Nat. Chem. 2015, 7, 153-159.
5 Catalano, L.; Perez-Estrada, S.; Terraneo, G.; Pilati, T.; Resnati, G.; Metrangolo, P.; Garcia-Garibay, M. A. Dynamic Characterization of Crystalline Supramolecular Rotors Assembled through Halogen Bonding. J. Am. Chem. Soc. 2015, 137, 15386-15389.
6 Li, M.; Hua, B.; Liang, H. Z.; Liu, J. Y.; Shao, L.; Huang, F. H. Supramolecular Tessellations via Pillar[n] arenes-Based Exo-Wall Interactions. J. Am. Chem. Soc. 2020, 142, 20892-20901.
7 Luo, D.; Tian, J.; Sessler, J. L.; Chi, X. Nonporous Adaptive Calix[4]pyrrole Crystals for Polar Compound Separations. J. Am. Chem. Soc. 2021, 143, 18849-18853.
8 Duggirala, N. K.; Perry, M. L.; Almarsson, O.; Zaworotko, M. J. Pharmaceutical Cocrystals: Along the Path to Improved Medicines. Chem. Commun. 2016, 52, 640-655.
9 Tian, S.; Bai, H.; Li, S.; Xiao, Y.; Cui, X.; Li, X.; Tan, J.; Huang, Z.; Shen, D.; Liu, W.; et al. Water-Soluble Organic Nanoparticles with Programable Intermolecular Charge Transfer for NIR-II Photothermal Anti-Bacterial Therapy. Angew. Chem. Int. Fd. 2021, 60, 11758-11762.
10 Lei, Y. L.; Liao, L. S.; Lee, S. T. Selective Growth of Dual-Color-Emitting Heterogeneous Microdumbbells Composed of Organic Charge-Transfer Complexes. J. Am. Chem. Soc. 2013, 135, 3744-3747.
11 Black, H. T.; Perepichka, D. F. Crystal Engineering of Dual Channel P/N Organic Semiconductors by Complementary Hydrogen Bonding. Angew. Chem. Int. Ed. 2014, 53, 2138-2142.
12 Ning, G.-H.; Cui, P.; Sazanovich, I. V.; Pegg, J. T.; Zhu, Q.; Pang, Z.; Wei, R.-J.; Towrie, M.; Jelfs, K. E.; Little, M. A.; et al. Organic Cage Inclusion Crystals Exhibiting Guest-Enhanced Multiphoton Harvesting. Chem 2021, 7, 3157-3170.
13 Zhang, J.; Xu, W.; Sheng, P.; Zhao, G.; Zhu, D. Organic Donor-Acceptor Complexes as Novel Organic Semiconductors. Acc Chem. Res. 2017, 50, 1654-1662.
14 Sun, Y.; Lei, Y.; Hu, W.; Wong, W.-Y. Epitaxial Growth of Nanorod Meshes from Luminescent Organic Cocrystals via Crystal Transformation. J. Am. Chem. Soc. 2020, 142, 7265-7269.
15 Braga, D.; Maini, L.; Grepioni, F. Mechanochemical Preparation of Co-Crystals. Chem. Soc. Rev. 2013, 42, 7638-7648.
16 Wang, Z.; Yu, F.; Chen, W.; Wang, J.; Liu, J.; Yao, C.; Zhao, J.; Dong, H.; Hu, W.; Zhang, Q. Rational Control of Charge Transfer Excitons toward High-Contrast Reversible Mechanoresponsive Luminescent Switching. Angew. Chem. Int. Ed. 2020, 39, 17580-17586.
17 Liu, Y. J., Li, A. S.; Xu, S. P.; Xu, W. Q.; Liu, Y.; Tian, W. J.; Xu, B. Reversible Luminescent Switching in an Organic Cocrystal: Multi-Stimuli-Induced Crystal-to-Crystal Phase Transformation. Angew. Chem. Int. Ed. 2020, 59, 15098-15103.
18 Yu, P.; Zhen, Y.; Dong, H.; Hu, W. Crystal Engineering of Organic Optoelectronic Materials. Chem 2019, 5, 2814-2853.
19 Park, S. K.; Kim, J. H.; Park, S Y. Organic 2D Optoelectronic Crystals: Charge Transport, Emerging Functions, and Their Design Perspective. Adv. Mater. 2018, 30, 1704759.
20 Huang, Y.; Wang, Z.; Chen, Z.; Zhang, Q. Organic Cocrystals: Beyond Electrical Conductivities and Field-Effect Transistors (FETs). Angew. Chem. Int. Ed. 2019, 58, 9696-9711.
21 Sun, L.; Wang, Y.; Yang, F.; Zhang, X.; Hu, W. Cocrystal Engineering: A Collaborative Strategy toward Functional Materials. Adv. Mater. 2019, 31, 1902328.
22 Coleman, L. B.; Cohen, M. J.; Sandman, D. J.; Yamagishi, F. G.; Garito, A F.; Heeger, A. J. Superconducting Fluctuations and the Peierls Instability in an Organic Solid. Solid State Commun. 1973, 12, 1125-1132.
23 Park, S. K.; Kim, J. H.; Ohto, T.; Yamada, R.; Jones, A. O. F.; Whang, D. R.; Cho, I.; Oh, S.; Hong, S. H.; Kwon, J. E.; et al. Highly Luminescent 2D-Type Slab Crystals Based on a Molecular Charge-Transfer Complex as Promising Organic Light-Emitting Transistor Materials Adv. Mater. 2017, 29, 1701346.
24 Tayi, A. S.; Shveyd, A. K.; Sue, A. C. H.; Szarko, J. M.; Rolczynski, B. S.; Cao, D.; Kennedy, T. J.; Sarjeant, A. A.; Stem, C. L.; Paxton, W. F.; et al. Room-Temperature Ferroelectricity in Supramolecular Networks of Charge-Transfer Complexes. Nature 2012, 488, 485-489.
25 Shin, M. H.; Lee, S. H.; Kang, B. J.; Jazbinsek, M.; Yoon, W.; Yun, H.; Rotermund, F.; Kwon, O. P. Organic Three-Component Single Crystals with Pseudo-Isomorphic Cocrystallization for Nonlinear Optics and Thz Photonics. Adv. Funct. Mater. 2018, 28, 1805257.
26 Bolton, O.; Lee, K.; Kim, H. J.; Lin, K. Y.; Kim, J. Activating Efficient Phosphorescence from Purely Organic Materials by Crystal Design. Nat. Chem. 2011, 3.205-210.
27 Gemen, J.; Bialek, M. J.; Kazes, M.; Shimon, L. J. W.; Feller, M.; Semenov, S. N.; Diskin-Posner, Y.; Oron, D.: Klajn, R. Ternary Host-Guest Complexes with Rapid Exchange Kinetics and Photoswitchable Fluorescence. Chem 2022, 8, 2362-2379.
28 Wang, Y.; Wu, H.; Hu, W.; Stoddart, J. F. Color-Tunable Supramolecular Luminescent Materials. Adv. Mater. 2022, 34, 2105405.
29 Park, S K.; Cho, I.; Gierschner, J.; Kim, J. H.; Kim, J. H; Kwon, J. E.; Kwon, O. K.; Whang, D. R.; Park, J.-H.; An, B.-K.; et al. Stimuli-Responsive Reversible Fluorescence Switching in a Crystalline Donor-Acceptor Mixture Film: Mixed Stack Charge-Transfer Emission Versus Segregated Stack Monomer Emission. Angew. Chem. Int. Ed. 2016, 55, 203-207.
30 Liu, G., Liu, J.; Ye, X., Nie, L., Gu, P.; Tao, X.; Zhang, Q. Self-Healing Behavior in a Thermo-Mechanically Responsive Cocrystal During a Reversible Phase Transition. Angew. Chem. Int. Ed. 2017, 56, 198-202.
31 Liu, Y.; Zeng, Q.; Zou, B.; Liu, Y.; Xu, B.; Tian, W. Piezochromic Luminescence of Donor-Acceptor Cocrystals: Distinct Responses to Anisotropic Grinding and Isotropic Compression. Angew. Chem. Int. Ed. 2018, 57, 15670-15674.
32 Borchers, T. H.; Topic, F.; Christopherson, J. C.; Bushuyev, O. S.; Vainauskas, J.; Titi, H M.; Friščić, T.; Barrett, C. J. Cold Photo-Carving of Halogen-Bonded Co-Crystals of a Dye and a Volatile Co-Former Using Visible Light. Nat. Chem. 2022, 14, 574-581.
33 Zhao, W.; He, Z., Tang, B. Z. Room-Temperature Phosphorescence from Organic Aggregates. Nat. Rev. Mater. 2020, 5, 869-885.
34 Zhuo, M.-P.; Yuan, Y.; Su, Y.; Chen, S.: Chen, Y.-T; Feng, Z.-Q.; Qu, Y.-K., Li, M.-D., Li, Y.; Hu, B.-W.; et al. Segregated Array Tailoring Charge-Transfer Degree of Organic Cocrystal for the Efficient Near-Infrared Emission Beyond 760 nm. Adv. Mater. 2022, 34, 2107169.
35 Li, S. Z.; Lu, B.; Fang. X. Y.; Yan, D. P. Manipulating Light-Induced Dynamic Macro-Movement and Static Photonic Properties within 1D Isostructural Hydrogen-Bonded Molecular Cocrystals. Angew. Chem. Int. Ed. 2020, 59, 22623-22630.
36 Qiao, B.; Hirsch, B. E.; Lee, S.; Pink, M.; Chen, C.-H.; Laursen, B. W.; Flood, A. H. Ion-Pair Oligomerization of Chromogenic Triangulenium Cations with Cyanostar-Modified Anions That Controls Emission in Hierarchical Materials. J. Am. Chem. Soc. 2017, 139, 6226-6233.
37 Benson, C. R.; Kacenauskaite, L.; VanDenburgh, K. L.; Zhao, W.; Qiao, B.; Sadhukhan, T.; Pink, M.; Chen, J.; Borgi, S.; Chen, C.-H.; et al. Plug-and-Play Optical Materials from Fluorescent Dyes and Macrocycles. Chem 2020, 6, 1978-1997.
38 Varughese, S. Non-Covalent Routes to Tune the Optical Properties of Molecular Materials. J. Mater. Chem. C 2014, 2, 3499-3516.
39 Yang, Y.-D.; Ji, X.; Lu, Z.-H.; Yang, J.; Gao, C.; Zhang, H.; Tang, B. Z.; Sessler, J. L.; Gong, H.-Y. Time-Dependent Solid-State Molecular Motion and Colour Tuning of Host-Guest Systems by Organic Solvents. Nat. Commun. 2020, 11, 77.
40 Liang, R.; Samanta, J.; Shao, B.; Zhang, M.; Staples, R. J.; Chen, A. D.; Tang, M.; Wu, Y.; Aprahamian, I.; Ke, C. A Heteromeric Carboxylic Acid Based Single-Crystalline Crosslinked Organic Framework. Angew. Chem. Int. Ed. 2021, 60, 23176-23181.
41 Yan, D.; Delori, A.; Lloyd, G. O.; Friscic, T.; Day, G. M.; Jones, W.; Lu, J.; Wei, M.; Evans, D. G.; Duan, X. A Cocrystal Strategy to Tune the Luminescent Properties of Stilbene-Type Organic Solid-State Materials. Angew. Chem. Int. Ed. 2011, 50, 12483-12486.
42 Wang, Y.; Wu, H.; Li, P.; Chen, S.; Jones, L. O.; Mosquera, M. A.; Zhang, L.; Cai, K.; Chen, H.; Chen, X.-Y.; et al. Two-Photon Excited Deep-Red and Near-Infrared Emissive Organic Co-Crystals. Nat. Commun. 2020, 11, 4633.
43 Bai, L.; Bose, P.. Gao, Q.; Li, Y.; Ganguly, R.; Zhao, Y. Halogen-Assisted Piezochromic Supramolecular Assemblies for Versatile Haptic Memory. J. Am. Chem. Soc. 2017, 139, 436-441.
44 Lei, Y.-L.; Jin, Y.; Zhou, D.-Y.; Gu, W.; Shi, X.-B.; Liao, L.-S.; Lee, S.-T. White-Light Emitting Microtubes of Mixed Organic Charge-Transfer Complexes. Adv. Mater. 2012, 24, 5345-5351.
45 Zhou, B.; Yan, L., Huang, J.; Liu, X.; Tao, L.; Zhang, Q. NIR II-Responsive Photon Upconversion through Energy Migration in an Ytterbium Sublattice. Nat. Photon. 2020, 14, 760-766.
46 Zhang, J.; Li, A.; Zou, H.; Peng, J.; Guo, J.; Wu, W.; Zhang, H.; Zhang, J.; Gu, X.; Xu, W.; et al. A “Simple” Donor-Acceptor AIEgen with Multi-Stimuli Responsive Behavior. Mater. Horiz. 2020, 7, 135-142.
47 Zhang, C.; Zhou, H. P.; Liao, L. Y.: Feng, W.; Sun, W.; Li, Z. X.; Xu, C. H.; Fang, C. J.; Sun, L D.; Zhang, Y. W.; et al. Luminescence Modulation of Ordered Upconversion Nanopatterns by a Photochromic Diarylethene: Rewritable Optical Storage with Nondestructive Readout. Adv. Mater. 2010, 22, 633-637.
48 Mase, K.; Sasaki, Y.; Sagara, Y.; Tamaoki, N.; Weder, C.; Yanai, N.; Kimizuka, N. Stimuli-Responsive Dual-Color Photon Upconversion: A Singlet-to-Triplet Absorption Sensitizer in a Soft Luminescent Cyclophane. Angew. Chem. Int. Ed. 2018, 57, 2806-2810.
49 Chen, G.; Qiu, H.; Prasad, P. N.; Chen, X. Upconversion Nanoparticles: Design, Nanochemistry, and Applications in Theranostics. Chem. Rev. 2014, 114, 5161-5214.
50 Deng, R.; Qin, F.; Chen, R.; Huang, W.; Hong, M.; Liu, X. Temporal Full-Colour Tuning through Non-Steady-State Upconversion. Nat. Nanotechnol. 2015, 10, 237-242.
51 Massaro, G.; Hernando, J.; Ruiz-Molina, D.; Roscini, C.; Latterini, L. Thermally Switchable Molecular Upconversion Emission. Chem. Mater. 2016, 28, 738-745.
52 Yao, W.; Tian, Q.; Wu, W. Tunable Emissions of Upconversion Fluorescence for Security Applications. Adv. Optical Mater. 2019, 7, 1801171.
53 Wu, Y.; Xu, J.; Qin, X.; Xu, J.; Liu, X. Dynamic Upconversion Multicolour Editing Enabled by Molecule-Assisted Opto-Electrochemical Modulation. Nat. Commun. 2021, 12, 2022.
54 Schneebeli, S. T.; Frasconi, M.; Liu, Z.; Wu, Y.; Gardner, D. M.; Strutt, N. L.; Cheng, C.; Carmieli, R.; Wasielewski, M. R.; Stoddart, J. F. Electron Sharing and Anion-π Recognition in Molecular Triangular Prisms. Angew. Chem. Int. Ed. 2013, 52, 13100-13104.
55 Beldjoudi, Y., Narayanan, A.; Roy, I.; Pearson, T. J., Cetin, M. M.; Nguyen, M. T., Krzyaniak, M. D.: Alsubaie, F. M.: Wasielewski, M. R.; Stupp, S. I.; et al. Supramolecular Tessellations by a Rigid Naphthalene Diimide Triangle. J. Am. Chem. Soc. 2019, 141, 17783-17795.
56 Ni, X.-L.; Xiao. X., Cong, H.; Zhu, Q.-J.; Xue, S.-F.; Tao, Z. Self-Assemblies Based on the “Outer-Surface Interactions” of Cucurbit[n]urils: New Opportunities for Supramolecular Architectures and Materials. Acc. Chem. Res. 2014, 47, 1386-1395.
57 Wu, H.; Wang, Y.; Jones, 1 O.; Liu, W.; Zhang, L.; Song, B.; Chen, X.-Y.; Stem, C. L.; Schatz, G. C.; Stoddart, J. F. Selective Separation of Hexachloroplatinate(IV) Dianions Based on Exo-Binding with Cucurbit[6]uril Angew. Chem. Int. Ed. 2021, 60, 17587-17594.
58 Wang, Y.; Wu, H.; Stoddart, J. F. Molecular Triangles: A New Class of Macrocycles. Acc. Chem. Res. 2021, 54, 2027-2039.
59 Vadehra, G. S.; Maloney, R P.; Garcia-Garibay, M. A.; Dunn, B. Naphthalene Diimide Based Materials with Adjustable Redox Potentials: Evaluation for Organic Lithium-Ion Batteries. Chem. Mater. 2014, 26, 7151-7157.
60 Suraru, S. L.; Würthner, F. Strategies for the Synthesis of Functional Naphthalene Diimides. Angew. Chem. Int. Ed. 2014, 53, 7428-7448.
61 Makarov, N. S.; Drobizhev, M.; Rebane, A. Two-Photon Absorption Standards in the 550-1600 nm Excitation Wavelength Range Opt. Express 2008, 16, 4029-4047.
62 Uejima, M.; Sato, T.; Tanaka, K.; Kaji, H. Enhancement of Fluorescence in Anthracene by Chlorination: Vibronic Coupling and Transition Dipole Moment Density Analysis. Chem. Phys. 2014, 430, 47-55.
63 Monson, P. R.; McClain, W. M. Complete Polarization Study of the Two-Photon Absorption of Liquid 1-Chloronaphthalene. J. Chem. Phys. 1972, 56, 4817-4825.
64 Lu, T.; Chen, F. Multiwfn: A Multifunctional Wavefunction Analyzer. J. Comput. Chem. 2012, 33, 580-592.
65 Lefebvre, C.; Rubez, G.: Khartabil, H.; Boisson, J.-C.; Contreras-Garcia, J.; Hënon, E. Accurately Extracting the Signature of Intermolecular Interactions Present in the NCI Plot of the Reduced Density Gradient Versus Electron Density. Phys. Chem. Chem. Phys. 2017, 19, 17928-17936.
66 Liu, C.; Minari, T.; Lu, X.; Kumatani, A.; Takimiya, K.; Tsukagoshi, K. Solution-Processable Organic Single Crystals with Bandlike Transport in Field-Effect Transistors. Adv. Mater. 2011, 23, 523-526.
67 Pawlicki, M.; Collins, H. A.; Denning, R G.; Anderson, H. L. Two-Photon Absorption and the Design of Two-Photon Dyes Angew. Chem. Int. Ed. 2009, 48, 3244-3266.
68 Drobizhev, M.; Makarov, N. S.; Tillo, S. E.; Hughes, T. E.; Rebane, A. Two-Photon Absorption Properties of Fluorescent Proteins. Nat. Methods 2011, 8, 393-399.
69 Sun, L.; Zhu, W.; Wang, W.; Yang, F.; Zhang, C.; Wang, S.; Zhang, X.; Li, R.; Dong, H.; Hu, W. Intermolecular Charge-Transfer Interactions Facilitate Two-Photon Absorption in Styrylpyridine-Tetracyanobenzene Cocrystals. Angew. Chem. Int. Ed. 2017, 56, 7831-7835.
70 Medishetty, R., Zareba, J. K.; Mayer, D.; Samoć, M.; Fischer, R. A. Nonlinear Optical Properties, Upconversion and Lasing in Metal-Organic Frameworks. Chem. Soc. Rev. 2017, 46, 4976-5004.
71 Schneebeli, S. T.; Frasconi, M.; Liu, Z.; Wu, Y.; Gardner, D. M.; Strutt, N. L.; Cheng, C.; Carmieli, R.; Wasielewski, M. R.; Stoddart, J. F. Electron Sharing and Anion—a Recognition in Molecular Triangular Prisms. Angew. Chem. Int. Ed. 2013, 52, 13100-13104.
72 Versluis, L.; Ziegler, T. The Determination of Molecular Structures by Density Functional Theory. The Evaluation of Analytical Energy Gradients by Numerical Integration. J. Chem. Phys. 1988, 88, 322-328.
73 Te Velde, G.; Bickelhaupt, F. M.; Baerends, E. J.; Fonseca Guerra, C.; Van Gisbergen, S. J. A.; Snijders, J. G.; Ziegler, T. Chemistry with ADF. J. Comput. Chem. 2001, 22, 931-967.
74 Van Lenthe, E.; Baerends, E. J. Optimized Slater-Type Basis Sets for the Elements 1-118. J. Comput. Chem. 2003, 24, 1142-1156.
75 Mosquera, M. A.; Chen, L. X.; Ratner, M. A.; Schatz, G. C Sequential Double Excitations from Linear-Response Time-Dependent Density Functional Theory. J. Chem. Phys. 2016, 144, 204105.
76 Mosquera, M. A.; Jones, L. O.; Kang, G.; Ratner, M. A.; Schatz, G. C. Second Linear Response Theory and the Analytic Calculation of Excited-State Properties. J. Phys. Chem. A 2021, 125, 1093-1102.
77 Adamo, C.; Barone, V. Toward Reliable Density Functional Methods without Adjustable Parameters: The PEB0 Model. J. Chem. Phys. 1999, 110, 6158-6170.
78 Silva, D. L., Krawczyk, P.; Bartkowiak, W.; Mendonga, C. R. Theoretical Study of One- and Two-Photon Absorption Spectra of Azoaromatic Compounds. J. Chem. Phys. 2009, 131, 244516.
79 Ohta, K., Antonov, L.; Yamada, S.; Kamada, K. Theoretical Study of the Two-Photon Absorption Properties of Several Asymmetrically Substituted Stilbenoid Molecules. J. Chem. Phys. 2007, 127, 084504.
80 Dolomanov, O. V.; Bourhis, L. J.; Gildea, R. J.; Howard, J. A. K.; Puschmann, H. Olex2: A Complete Structure Solution, Refinement and Analysis Program. J. Appl. Cryst. 2009, 42, 339-341.
81 Sheldrick, G. Shelxt—Integrated Space-Group and Crystal-Structure Determination. Acta Cryst. A 2015, 71, 3-8.
82 Sheldrick, G. A Short History of Shelx. Acta Cryst. A 2008, 64, 112-122.
83 Thorn, A, Dittrich, B.; Sheldrick, G. M Enhanced Rigid-Bond Restraints. Acta Cryst. A 2012, 68, 448-451.
84 Lefebvre, C.; Rubez, G.; Khartabil, H.; Boisson, J.-C; Contreras-Garcia, J.; Hénon, E Accurately Extracting the Signature of Intermolecular Interactions Present in the NCI Plot of the Reduced Density Gradient Versus Electron Density. Phys. Chem. Chem. Phys. 2017, 19, 17928-17936.
85 Lu, T.; Chen, F. Multiwfn: A Multifunctional Wavefunction Analyzer. J. Comput. Chem. 2012, 33, 580-592.
86 Pettersen, E. F.; Goddard. T. D.; Huang, C. C.; Couch, G. S.; Greenblatt. D. M., Meng, E. C., Ferrin, T. E. UCSF Chimera-A Visualization System for Exploratory Research and Analysis. J. Comput. Chem. 2004, 25, 1605-1612.

### Miscellaneous

Unless otherwise specified or indicated by context, the terms “a”, “an”, and “the” mean “one or more.” For example, “a molecule” should be interpreted to mean “one or more molecules.” As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein. Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A charge transfer cocrystal comprising an electron acceptor macrocycle and an electron donor.

2. The charge transfer cocrystal of claim 1, wherein the charge transfer cocrystal upconverts two or more absorbed photons.

3. The charge transfer cocrystal of any one of the preceding claims, wherein the charge transfer cocrystal is color-tunable by substituting the electron donor for a second, different electron donor.

4. The charge transfer cocrystal of any one of the preceding claims, wherein the macrocycle is a triangular macrocycle.

5. The charge transfer cocrystal of claim 4, wherein the macrocycle is a naphthalenediimide-based triangular macrocycle (NDI-Δ) or a derivative thereof.

6. The charge transfer cocrystal of any one of the preceding claims, wherein the electron donor is a substituted anthracene or a substituted naphthalene.

7. The charge transfer cocrystal of any one of the preceding claims, wherein the electron donor is an anthracene substituted with one or more halogen or a naphthalene substituted with one or more halogen.

8. The charge transfer cocrystal of any one of the preceding claims, wherein the electron donor is an anthracene substituted with one or more Cl or a naphthalene substituted with one or more Cl.

9. The charge transfer cocrystal of any one of the preceding claims, wherein the electron donor is 9, 10-dichloroanthracene (DCA) or 1-chloronaphthalene (CN).

10. The charge transfer cocrystal of any one of the preceding claims, wherein the cocrystal is DCA·NDI-Δ or CN·NDI-Δ.

11. A method for upconverting photons, the method comprising irradiating a charge transfer cocrystal with photons at a first energy state, the charge transfer cocrystal comprising an electron acceptor macrocycle and an electron donor, whereby photons at a second energy state are emitted, the second energy state being higher than the first energy state.

12. The method of claim 11, wherein the macrocycle is a triangular macrocycle.

13. The method of any one of claims 11-12, wherein the macrocycle is a naphthalenediimide-based triangular macrocycle (NDI-Δ) or a derivative thereof.

14. The method of any one of claims 11-13, wherein the electron donor is 9, 10-dichloroanthracene (DCA) or 1-chloronaphthalene (CN).

15. The method of any one of claims 11-14, wherein the cocrystal is DCA·NDI-Δ or CN·NDI-Δ.

16. A method for tuning color of light emission, the method comprising
providing a first charge transfer cocrystal comprising an electron acceptor macrocycle and a first electron donor; and
at least partially substituting the first electron donor of the first charge transfer cocrystal with a second, different electron donor to produce a second charge transfer cocrystal,
wherein the first charge transfer cocrystal when irradiated emits at a first wavelength; and wherein the second charge transfer cocrystal when irradiated emits a second, different wavelength.

17. The method of claim 16, further comprising substituting the second electron donor of the second charge transfer cocrystal with the first electron donor, thereby restoring the first charge transfer cocrystal.

18. The method of any one of claims 16-17, further comprising irradiating the second charge transfer cocrystal.

19. The method of any one of claims 16-18, further comprising irradiating the first and the second charge transfer cocrystals.

20. The method of any one of claims 16-19, wherein the macrocycle is a triangular macrocycle.

21. The method of any one of claims 16-20, wherein the macrocycle is a naphthalenediimide-based triangular macrocycle (NDI-Δ) or a derivative thereof.

22. The method of any one of claims 16-21, wherein one of the first and the second electron donor is 9, 10-dichloroanthracene (DCA) and the other is 1-chloronaphthalene (CN).

23. The method of any one of claims 16-22, wherein one of the first and the second cocrystal is DCA·NDI-Δ and the other is CN·NDI-Δ.

24. A crystalline luminescent material comprising the charge transfer cocrystal of claim 1.

25. The crystalline luminescent material of claim 24, which is a wavelength tunable upconversion-emission material.

26. The crystalline luminescent material of any one of claims 24-25, wherein the cocrystal is DCA·NDI-Δ, CN·NDI-Δ, or a combination thereof.

27. An apparatus comprising the charge transfer cocrystal of claim 1 or the crystalline luminescent material of claim 24.

28. The apparatus of claim 27, which is color-tunable.

29. The apparatus of claim 27, which is a color-tunable upconversion laser, a high-resolution display, or a multicolor imaging system.

30. The apparatus of claim 27, comprising a dual-color switch formed by two different charge transfer cocrystals of claim 1.

31. The apparatus of claim 30, wherein the two cocrystals exhibit two-photon excited fluorescence.

32. The apparatus of claim 27, wherein the cocrystal is DCA·NDI-Δ, CN·NDI-Δ, or a combination thereof.
